(19) United States
(12) Patent Application Publication
(10) Pub. No.: US 2025/0011725 A1
(43) Pub. Date: Jan. 9, 2025

(54) TISSUE-ENGINEERED POROUS CERAMIC TEMPLATE AND USES THEREOF

(71) Applicant: Osteogene Bio Inc., Norwood, NJ (US)

(72) Inventor: Daniel S. Oh, Northvale, NJ (US)

(73) Assignee: Osteogene Bio Inc., Norwood, NJ (US)

(21) Appl. No.: 18/763,772

(22) Filed: Jul. 3, 2024

Related U.S. Application Data
(60) Provisional application No. 63/525,091, filed on Jul. 5, 2023.

(51) Int. Cl. C12N 5/09; C12M 1/00; C12M 1/12

(52) U.S. Cl. CPC C12N 5/0693; C12M 25/14; C12M 29/10; C12N 2513/00; C12N 2533/14

(57) ABSTRACT

A tissue engineered three-dimensional model and related processes and apparatus. The model includes a three-dimensional porous ceramic template including a plurality of primary macro-pores; and cells cultured in the three-dimensional porous ceramic template, wherein at least some of the cells form one or more three-dimensional cellular matrices in the primary macro-pores. A process for preparing a tissue engineered three-dimensional model includes the steps of preparing the three-dimensional porous ceramic template, including a plurality of primary macro-pores; and culturing cells in the three-dimensional porous ceramic template, wherein at least some of the cells form one or more three-dimensional cellular matrices in the primary macro-pores.

### CROSS-REFERENCE TO RELATED APPLICATION

This application claims priority to U.S. Provisional Patent Application Ser. No. 63/525,091, filed Jul. 5, 2023, the entire disclosure of which is incorporated herein by reference.

### FIELD

The present disclosure generally relates to a three-dimensional porous ceramic template and uses thereof and, more particularly, to a three-dimensional porous ceramic template having an interconnected pore structure suitable for the co-culture of normal or bone cells and/or cancer cells and uses of same as a three-dimensional cancer culture model.

### BACKGROUND

Two-dimensional (2D) cancer cell culture models are available to investigate disease mechanisms and to screen therapies. While these models have contributed information about cancer biology, these simplistic models fail to adequately model the in vivo environment such as a three-dimensional cancer model. Approximately 90% of potentially promising preclinical drugs, in all therapeutic classes, fail to result in efficacious human treatments, wasting vast amounts of time and money and, ultimately, delaying the discovery of successful interventions. Two-dimensional tissue culture models lack realistic complexity, while animal models are expensive, time consuming, and too frequently fail to reflect human tumor biology. These 2D culture systems do not reflect the true three-dimensional (3D) microenvironment present in human tissues and/or tumors, whereby cell-cell and cell-extracellular matrix (ECM) interactions occur.

3D, spheroidal, cancer cell culture models are available to investigate disease mechanisms and to test therapies. While these models provide a 3D microenvironment like in-vivo, they lack realistic complexities, such as 3D volume, cell-cell, cell-extracellular matrix (ECM) interactions, and hypoxic conditions, like in the 2D culture systems discussed above.

Thus, there remains a need in the art for a 3D model having a biological microenvironment adapted for simulating cell-cell, cell-extracellular matrix (ECM) interactions, and hypoxic conditions present in human tissues and/or tumors and for promoting cancer cell proliferation, motility, and differentiation.

### SUMMARY

The present disclosure relates to systems, methods, and apparatus for providing a tissue engineered three-dimensional model. The three-dimensional model includes tumor or cancer cells, normal cells, and a porous ceramic template. The subject matter of the present disclosure is suitable for testing therapeutic agents for various purposes (e.g., for investigating their effect on the tumor and/or normal cells for devising a medical or cancer treatment customized for or personalized to an individual patient). In addition, it is suitable for testing other therapies that require a 3D microenvironment simulating in vivo cancer growth.

In one embodiment, a three-dimensional porous ceramic template includes primary macro-pores defined by a plurality of struts, secondary micro-channels formed in the struts, and tertiary sub-micro holes formed in the surfaces of the struts. In one embodiment, one or more of the primary macro-pores may be connected to or interconnected with one or more of the secondary micro-channels and/or one or more of the tertiary sub-micro holes. The primary macro-pores, which (or at least some of which) are connected to or interconnected with each other, are about 100-600 μm in diameter and are formed throughout the template for providing spaces for cells to migrate deep into the template and to grow throughout the template. In one embodiment, one or more of the secondary micro-channels may be connected to or interconnected with one or more of the primary macro-pores and/or one of more of the tertiary sub-micro holes. The secondary micro-channels, which (or at least some of which) are connected to or interconnected with each other, are about 20-70 μm in diameter and are formed for providing continuous fluid flow to supply oxygen and nutrients to the cells and additional surface areas for cell attachment. In one embodiment, one or more of the tertiary sub-micro holes may be connected to or interconnected with one or more of the primary macro-pores and/or one or more of the secondary micro-channels. The tertiary sub-micro holes, at least some of which may be connected to or interconnected with each other, are about 80-400 nm in diameter and are formed in the surfaces of the template, such as the surfaces of the struts, to encourage cells to anchor.

In some embodiments, the porous ceramic template is substantially cylindrical. In some other embodiments, the porous ceramic template has a diameter about 5-10 mm and/or height about 5-10 mm. In other embodiments, the porous ceramic template may be provided with other diameters, heights, sizes, dimensions, and/or shapes.

In some embodiments, the porous ceramic template is made from suitable ceramic materials, including, but not limited to, alumina, zirconia, titania, glass oxide, calcium phosphate-based oxide such as hydroxyapatite, tricalcium phosphate, and/or magnesium/strontium/zinc-substituted calcium phosphate-based oxide.

In some embodiments, the porous ceramic template has a plurality of primary cells and/or a plurality of secondary cells seeded thereon or therein. To create a co-culture model, the primary cells include normal cells, such as osteoblast precursors, osteoblasts, osteoclasts, fibroblasts, muscle cells, bone marrow cells, and/or mesenchymal stem cells. In other embodiments, the primary cells may include other normal cells. In some embodiments, the secondary cells include tumor and/or cancer cells, such as osteosarcoma, chondrosarcoma, Ewing's sarcoma, fibrosarcoma, and/or breast cancer cell. In other embodiments, the secondary cells may include other cancel and/or tumor cells. In some embodiments, the primary cells are introduced into the porous ceramic template to establish a normal cellular microenvironment. After establishing a normal cellular microenvironment, the secondary cancer cells are introduced into the porous ceramic template to mimic an in-vivo 3D cancer model.

Another embodiment of the invention includes a process for preparing a tissue engineered three-dimensional model. The process includes the steps of: preparing a three-dimensional porous ceramic template including a plurality of primary macro-pores; and culturing cells in the three-dimensional porous ceramic template, wherein at least some of the cells form one or more three-dimensional cellular matrices in the primary macro-pores.

In some embodiments, a bioreactor is provided. The bioreactor includes a column connected with a peristaltic perfusion pump operable to circulate a culture medium. In some embodiments, the culture medium comprises a pharmaceutical chemotherapeutic agent and/or another therapeutic agent.

Another embodiment of the invention includes an apparatus that includes a tissue engineered three-dimensional model, including a three-dimensional porous ceramic template including a plurality of primary macro-pores, and cells cultured in the three-dimensional porous ceramic template, wherein at least some of the cells form one or more three-dimensional cellular matrices in the primary macro-pores. The apparatus further includes a bioreactor system including a culture column configured to receive the three-dimensional porous ceramic template, a culture medium vessel operably connected to the culture column, and a peristaltic perfusion pump operably connected to the culture column and to the culture medium vessel, wherein the peristaltic perfusion pump is configured to apply a dynamic culture system within the culture column.

It is to be understood that both the foregoing general description and the following detailed description are exemplary and are intended to provide a further explanation of the disclosed subject matter. The accompanying drawings, which are incorporated in and constitute part of this specification, are included to illustrate and provide a further understanding of the systems, methods, and apparatus of the disclosed subject matter. Together with the description and drawings explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part. Similar reference numerals (differentiated by the leading numeral) may be provided among the various views and figures presented herein to denote functionally corresponding, but not necessarily identical structures.

The patent or application file contains at least one drawing executed in color. Copies of this patent or patent application publication with color drawing(s) will be provided by the Office upon request and payment of the necessary fee.

FIGS. 1A-1C illustrate an exemplary porous ceramic template (“PCT”) according to embodiments of the present disclosure.

FIG. 2 is an enlarged view of a section of the PCT shown in FIG. 1A.

FIG. 3 illustrates primary macro-pores, secondary micro-channels, and tertiary sub-micro holes of an exemplary PCT template according to embodiments of the present disclosure.

FIG. 4 is a schematic illustration of interconnected secondary micro-channels and fluid flow therethrough according to embodiments of the present disclosure.

FIG. 5 is a schematic illustration of secondary micro-channels and tertiary sub-micro holes according to embodiments of the present disclosure.

FIG. 6 illustrates schematic illustrations of PCTs without and with micro-channels and a surface area comparison table.

FIG. 7 (FIGS. 7A-C) is a schematic illustrating an example of a bi-layered and multi structural bone-like three-dimensional calcium phosphate scaffold for bone augmentation. FIG. 7A shows the longitudinal cross section of a bi-layered scaffold with the porous outer cortical shell (1) and porous inner trabecular core structure (2). FIG. 7B shows cross-section of a bi-layered scaffold with the porous outer cortical shell (1) and porous inner trabecular core structure (2). FIG. 7C shows the cross-section of a dense calcium phosphate-coated strut (3) with the presence of triangular secondary micro-channel (4) within the strut.

FIG. 8 is a schematic showing one example of the making of a bi-layered templates, with the inner trabecular core sponge snuggly fitted into the outer cortical shell sponge.

FIG. 9 is a temperature/time graph showing an exemplary 8-step sintering profile of a calcium phosphate-coated polyurethane sponge after the first calcium phosphate coating procedure.

FIG. 10 temperature/time graph showing an exemplary 5-step sintering profile for second coated calcium phosphate scaffold sintering procedure.

FIG. 11 is a flow chart showing a process for producing silver-doped hydroxyapatite sol.

FIG. 12 is a temperature/time graph showing an exemplary 3-step sintering profile of a scaffold after coating the scaffold with or without silver- or zinc-doped calcium phosphate sol.

FIG. 13 includes two representative scanning electron micrographs showing (a) the untreated surface of the sponge template, and (b) the polyurethane sponge surface after a 20 minute treatment in 10% NaOH. Microcracks on the treated sponge surface are observed and these cracks allow the nucleation of calcium phosphate coatings and ensure the uniformity of the coating on the sponge surface.

FIG. 14 is a TG/DTA curve of a polyurethane sponge template.

FIG. 15 is a representative scanning electron micrograph showing a dense and smooth scaffold surface after sintering (magnification×5,000). Grain boundaries of calcium phosphate on the scaffold surface are also observed.

FIG. 16 includes representative scanning electron micrographs (SEM) of a scaffold of the present invention after 2^{nd} sintering showing (a) cross-section of the interconnecting secondary micro-channels within the strut, (b) high magnification (magnification×1,500) of the strut showing the triangular secondary micro-channel, with length of each side ranging from 30 μm to 120 μm, and (c) a primary pore having diameter ranging from 150 μm to 750 μm (magnification×150).

FIG. 17 includes representative scanning electron micrographs (SEM) showing the surface and thickness of a strut after (a) 1^{st} time coating and sintering, and (b) 2^{nd} time coating and sintering.

FIG. 18 is a representative cross section of a calcium phosphate scaffold infiltrated with bone tissue and vascular in-growth after 12 weeks post-surgery, 200× (S=scaffold, V=vessel). This histological section is viewed under a phase contrast microscope.

FIG. 19 is a flow chart showing a non-limiting method of the present invention.

FIG. 20 shows scanning electron microscopy of different cross sections of one scaffold of the present invention showing (a) an outer cortical shell with micro-channel; (b) an inner layer of the strut; (c) a roughed surface of the strut; and (d) a cross section of the hollow strut.

FIGS. 21A-XX include a pictorial description of a method of preparing a scaffold of the present invention, as follows:

FIG. 21A shows polyurethane (PU) sponges that may be used to produce interconnected porous CaP scaffolds.

FIGS. 21C-G show the following steps: To change PU sponge surface characteristics from hydrophobic to hydrophilic and increase wettability, a prepared PU sponge may be ultrasonically treated in 10% NaOH solution for 20-30 minutes prior to use. Cleaning with flowing water for 15-20 minutes followed. During cleaning, the sponge may be squeezed and expanded 3-4 times to rinse residual NaOH inside the PU sponge. Ultrasonically cleaning with distilled water for 15-20 minutes may follow. After removing water with, e.g., a paper towel, the sponge may be placed in a 60-80° C. oven until completely dry (e.g., 80° C. for 5 hours).

FIG. 21H shows that after completely drying the core sponge (cancellous bone part), it may be plugged into an outside shell (cortical bone part) porous sponge or solid shell depending on what is desired in the final structure and application.

FIGS. 21J-K show that to make a slip casting slurry, a binder is preferably added to the dispersion. The binders may be carboxymethylcellulose (CMC), polyvinyl alcohol, starch, sodium silicate, polyvinyl butyral, methacrylate emulsion, water soluble polyacrylate, polyacrylic acid, polyethylene glycol, etc. A particularly preferable binder, in certain embodiments, is carboxymethylcellulose and sodium silicate. The amount of carboxymethylcellulose added is preferably 5-10% by mass and sodium silicate solution added is preferably 2-5% by mass based on 100% by mass of calcium phosphate powder. After adding carboxymethylcellulose into distilled water, further stirring is conducted until completely dissolved then add sodium silicate solution and stirring.

FIG. 21L shows that to keep homogeneity and prevent rapid sedimentation of calcium phosphate powder, ammonium polyacrylate may be added (e.g., 5-10% by mass based on 100% by mass of powder for dispersant).

FIG. 21M shows that to prevent cracks due to rapid drying during the drying process, N,N-dimethylformamide may be added (e.g., 10-15% by mass based on 100% by mass of powder for drying agent).

FIG. 21N shows that to make the calcium phosphate slurry, calcium phosphate powder is slowly spread into the solution.

FIG. 21O shows that after adding the calcium phosphate powder, further stirring is conducted and also the slurry is heated at 40-50° C. for water evaporation during stirring until the powder/liquid ratio is 0.3-0.4.

FIG. 21P shows that calcium phosphate slurry may be poured plaster cast mold for casting solid outside shell. After the slurry is poured, the plaster cast mold is rotated to obtain a homogeneously thick solid outside shell. This may be repeated several times until the desired outside shell thickness is achieved.

FIG. 21Q shows that after the solid outside green body shell is completely casted, it may be dried at 30° C. and above 80% humidity in a chamber. The green body may then be separated from the plaster cast mold and dried at 25° C., under 30% humidity air conditions, for 6-24 hours depending on green body size. It is then placed into a furnace for sintering.

FIG. 21R shows a first step of heating until 600° C.

FIGS. 21T-U show that to make the 1st coating calcium phosphate paste, a binder is preferably added to the dispersion. Such binders are described herein. After adding polyvinyl alcohol into distilled water, further stirring is conducted until all is completely dissolved; then sodium silicate solution is added with continued stirring.

FIGS. 21V-W show that the amount of carboxymethylcellulose added is preferably 3-5% by mass. After adding carboxymethylcellulose into solution, further stirring is conducted until all is completely dissolved, then ammonium polyacrylate is added (3-5% by mass based on 100% by mass of calcium phosphate powder) with stirring.

FIG. 21X shows that to prevent cracks due to rapid drying during the drying process, N,N-dimethylformamide may be added (e.g., 5-10% by mass amount based on 100% by mass of powder for drying agent).

FIG. 21Y shows that the calcium phosphate slurry may be made by slowly spreading calcium phosphate powder into the solution.

FIG. 21Z shows the calcium phosphate paste as a first coating and sintering.

FIG. 21AA shows that after adding the calcium phosphate, powder further stirring is conducted and the slurry is heated at 40-50° C. for water evaporation during stirring until the powder/liquid ratio is 1.0-1.25. If stirrer bar is stopped during stirring, stir with a Teflon bar until to get the desired powder/liquid ratio.

FIG. 21BB shows that the pre-treated bi-layered PU sponge is immersed in the calcium phosphate paste then squeezed and expanded 5-7 times using a Teflon bar. Excess paste is removed with air to avoid the primary pores being filled with paste. The homogeneous coating may be examined using a stereo microscope.

FIGS. 21CC-DD show that after examining the homogeneous coating, the pre-formed scaffold is dried at 30° C., 50-70% humidity. Then the pre-dried calcium phosphate coated mono or bi-layered pre-formed scaffold is dried at 25° C., under 30% humidity air conditions, for 6-24 hours depending on the pre-formed size. After completely drying, the pre-formed scaffold is put into a furnace for 1st sintering.

FIG. 21EE shows the calcium phosphate slurry as a second coating, and dissolving the polyvinyl alcohol (PVA).

FIG. 21FF-GG show that to make the 1st coating calcium phosphate paste, a binder is preferably added to the dispersion. Such binders are described herein. After adding polyvinyl alcohol into distilled water, further stirring is conducted until all is completely dissolved; then sodium silicate solution is added with continued stirring.

FIGS. 21HH-II show that the amount of carboxymethylcellulose added is preferably 3-5% by mass. After adding carboxymethylcellulose into solution, further stirring is conducted until all is completely dissolved, then ammonium polyacrylate is added (3-5% by mass based on 100% by mass of calcium phosphate powder) with stirring.

FIG. 21JJ shows that to prevent cracks due to rapid drying during the drying process, N,N-dimethylformamide may be added (e.g., 5-10% by mass amount based on 100% by mass of powder for drying agent).

FIG. 21KK shows that the calcium phosphate slurry may be made by slowly spreading calcium phosphate powder into the solution.

FIG. 21LL shows the calcium phosphate slurry as a second coating with sintering.

FIG. 21MM shows that after adding the calcium phosphate powder, further stirring is conducted and the slurry is heated at 40-50° C. for water evaporation during stirring until powder/liquid ratio is 0.3-0.4.

FIG. 21NN shows that the 1st sintered mono or bi-layered scaffold is immersed in the calcium phosphate slurry and taken out after 5 seconds. Excess slurry is removed using air to avoid filling the primary pores with slurry.

FIG. 21OO shows that the 2nd time coated mono or bi-layered scaffold is centrifuged to remove the 2nd excess slurry and to obtain a homogeneous coating for 10-20 seconds at 1000-2000 rpm, depending on scaffold size and slurry viscosity.

FIGS. 21PP-QQ show that after centrifuging, the scaffold is dried at 25° C., under 30% humidity air conditions, for 6-24 hours depending on the pre-formed size. After completely drying the 2nd time coated scaffold, it is placed into a furnace for 2nd sintering.

FIG. 21RR shows the antibacterial calcium phosphate doped with silver or zinc can be synthesized using the Sol-Gel method.

FIG. 21SS shows that the silver- or zinc-doped calcium phosphate sol is prepared by synthesizing the calcium (Ca), silver (Ag) precursor and the phosphorus (P) precursor.

FIG. 21TT shows that the silver- or zinc-doped calcium phosphate sol is then synthesized by reacting calcium and phosphorus precursors for a period of 1 to 2 hours and with vigorous stirring. The reaction is performed under an argon atmosphere.

FIG. 21UU shows that the synthesized silver- or zinc-doped calcium phosphate sol is then filtrated through a 0.20 μm to 0.45 μm syringe filter, followed by aging at temperatures ranging from 40° C. to 80° C. and for a period ranging from 12 to 204 hours.

FIG. 21VV shows that the fabricated porous calcium phosphate scaffolds are then immersed in the aged calcium phosphate sol doped with or without silver or zinc. After immersing for 5 to 10 seconds, the scaffold is then removed from the sol and air blown to unclog the pores.

FIG. 21WW shows that the scaffolds are centrifuged to remove excess sol.

FIG. 21XX shows that the calcium phosphate sol coated scaffold is then baked and dried in an oven at temperatures ranging from 50° C. to 100° C. and for a period ranging from 3 to 8 hours. After they are completely dried, the calcium phosphate sol-coated scaffolds are then heat-treated at temperatures ranging from 600° C. to 700° C. using a muffle furnace in air for a period ranging from 1 hour to 5 hours.

FIG. 22 shows a curved custom made scaffold configured to fit the shape, anatomical structure and size of rabbit tibia.

FIG. 23A shows granules of the present invention.

FIG. 23B shows radiographic imaging of the granules of FIG. 23A following placement in a bony defect.

FIG. 24 shows fabrication procedures for scaffolds with micro-channels and nano-pores according to an embodiment of of the present invention, including: (A) the treatment of PU template for surface modification, (B) the coating step of PU template with HA slurry, and (C, D) the heat treatment of HA coated PU template using high temperature furnace.

FIG. 25 shows the overall bone-like template fabrication protocol from the pre-treatment of PU sponge (P1) to the final heat treatment (P7). Keeping the precise sintering profile after P7 is crucial in achieving favorable mechanical strength.

FIG. 26 shows representative stereo microscope (AmScope; SM-2TZ-M) images (×4) of an 80 ppi sized PU sponge (left), HA coated and dried BMT (middle), and sintered BMT (right). (Dimension: 3 cm in height×4 cm in length×1 cm in width).

FIG. 27 shows SEM and micro-CT images of a biogenic template: (A) an overall image of a biogenic template, (B, C, D) images for micro-channels. In order to highlight clear micro-channels in the trabeculae, the template was granulated.

FIG. 28 shows computational calculations of capillary action with different channel diameters. Within the same time period (0.4 ms), while the largest capillary (d=300 μm: refers to primary-pore) absorbed the medium (blue) up to 0.16 mm in height, the smallest capillary (d=30 μm: refers to micro-channel) absorbed the medium up to 0.415 mm in height.

FIG. 29 shows differences of absorption capabilities of capillary action based on different sizes of primary-pores and micro-channels (primary-pore size refers to the average diameter: 60 ppi≈470 μm, 80 ppi≈320 μm, 100 ppi≈200 μm). The yellow lines represent the capillary action induced by the combination of primary-pores and micro-channels. The red lines represent the capillary action induced by mainly micro-channels exhibited in each trabecula. As shown in (F), the 100 ppi template induced the strongest capillary action, resulting in the complete saturation of the template within 39 sec. The 80 ppi and 60 ppi templates were tested thereafter. (B) 0 sec, (C) 0.5 sec, (D) 1.5 sec, (E) 17.0 sec, (F) 39.0 sec, (G) 50.0 sec, and (H) 1 min 18 sec after immersion. (Template dimension: 1 cm×1 cm×4 cm in height cuboidal).

FIG. 30 shows the ingress and immigration of cells from the seeded wells (part I) to unseeded wells (part V) through the biogenic templates induced by capillary action. The initially seeded cells reached the end of the unseeded leg (part V) immediately after full saturation. After 3 days, the confluence of the cells was evident throughout the entire template. After 7 days, spatiotemporal collagen matrix formation occurred within the cell populations (H&E stain). (Template dimension: 3 cm in height×4 cm in length×1 cm in width).

FIG. 31 illustrates a static culture system (FIG. 31A) and a dynamic culture system (FIG. 31B) including a bioreactor for normal cell 3D culture according to embodiments of the present disclosure.

FIG. 32 illustrates a static culture system (FIG. 32A) and a dynamic culture system (FIG. 32B) including a bioreactor for 3D co-culture associated with normal cells and tumor cells according to embodiments of the present disclosure.

FIG. 33 illustrates a dynamic culture system including a bioreactor for therapeutic treatment of 3D cancer model mimicry in vivo microenvironments according to embodiments of the present disclosure.

FIG. 34 illustrates a co-cultured 3D cancer model inside of a PCT according to embodiments of the present disclosure.

FIG. 35 illustrates an exemplary cylindrical 3D cancer culture model according to embodiments of the present disclosure.

FIG. 36 illustrates a comparison of cell viability among 2D static culture on a culture plate without a porous ceramic template (PCT), 3D static culture using a PCT for designated time, and 3D dynamic culture using a PCT for designated time.

FIG. 37 illustrates a comparison of survival index of co-cultured cells on a culture plate for 2D static culture without a porous ceramic template (PCT), 3D static culture using a PCT, and 3D dynamic culture using a PCT after treatment with doxorubicin (DOX: therapeutic drug) for designated time.

### DETAILED DESCRIPTION OF EXEMPLARY EMBODIMENTS

Reference will now be made in detail to exemplary embodiments of the disclosed subject matter, examples of which are illustrated in the accompanying drawings. Methods and corresponding steps of the disclosed subject matter will be described in conjunction with the accompanying drawings.

A co-cultured 3D microenvironment cancer model according to embodiments of the present disclosure is suitable for the screening and/or testing of chemotherapeutic drugs or other therapeutic agents. According to various embodiments of the present disclosure, an engineered 3D culture platform is provided that comprises a porous ceramic template (PCT) having a three-leveled micro-architecture. The PCT includes interconnected primary macro-pores that mimic those in trabecular bone in function and/or structure. In some embodiments, the macro-pores are about 100-600 μm in diameter. The macro pores provide a primary space for co-cultured cells to grow three-dimensionally to mimic in vivo cancer microenvironments. The PCT also includes micro-channels within each trabecular-like structure. In some embodiments, the micro-channels are about 20-70 μm in diameter. The micro-channels are adapted to permit or provide continuous fluid flow even after the primary macro-pores are filled by 3D cancer model. More particularly, the micro-channels provide secondary, additional surfaces for cells to anchor. Because of the engineered double surfaces of the PCT, the proliferation rate is dramatically enhanced, compared to regular bone tissue scaffold that includes decellularized human bone scaffold. The elevated proliferation rate is closely related with a corresponding local oxygen concentration. The PCT also contains sub-micro holes on its outer surfaces. In some embodiments, the sub-micro holes are about 80-400 nm in diameter.

### Porous Ceramic Template (PCT)

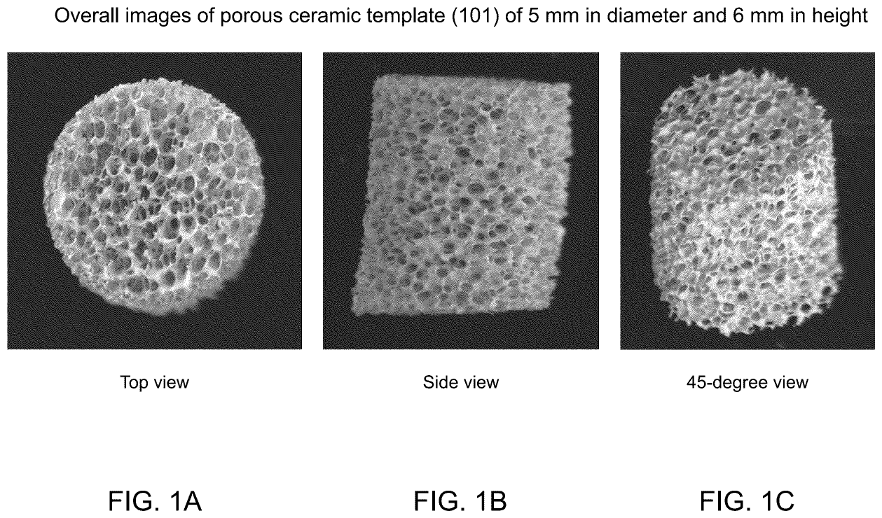

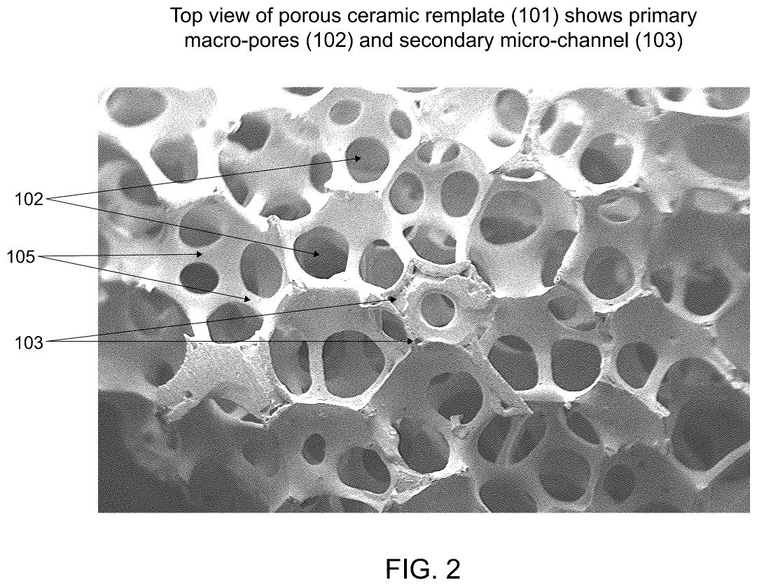

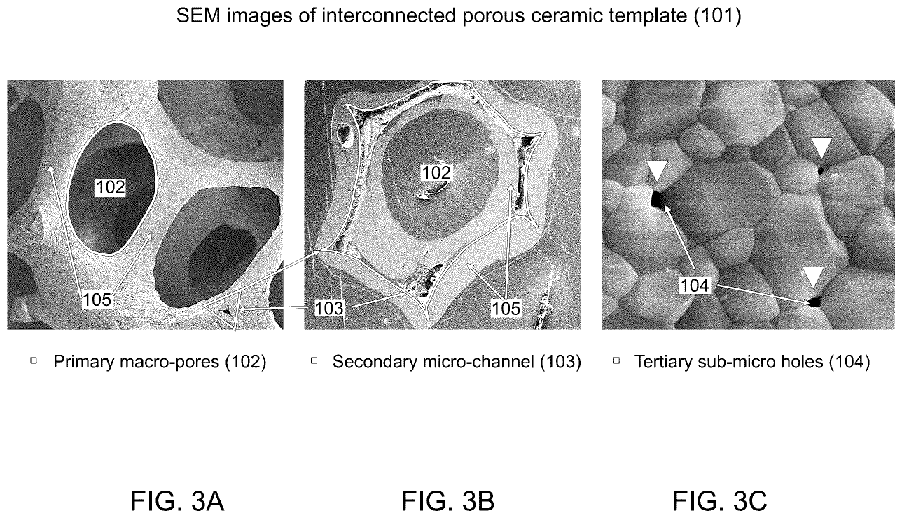

Referring now to FIGS. 1A-3C, an exemplary PCT 101 is depicted. FIGS. 2 and 3A depict a plurality of primary macro-pores 102. FIG. 3B is a zoomed-in view of a cross section of a macro-pore of the PCT illustrated in FIG. 3A, showing a primary macro-pore 102 and secondary micro-channels 103 (see also FIG. 2). FIG. 3C provides a further zoomed-in view of a surface of the PCT shown in FIG. 3A, showing tertiary sub-micro holes 104.

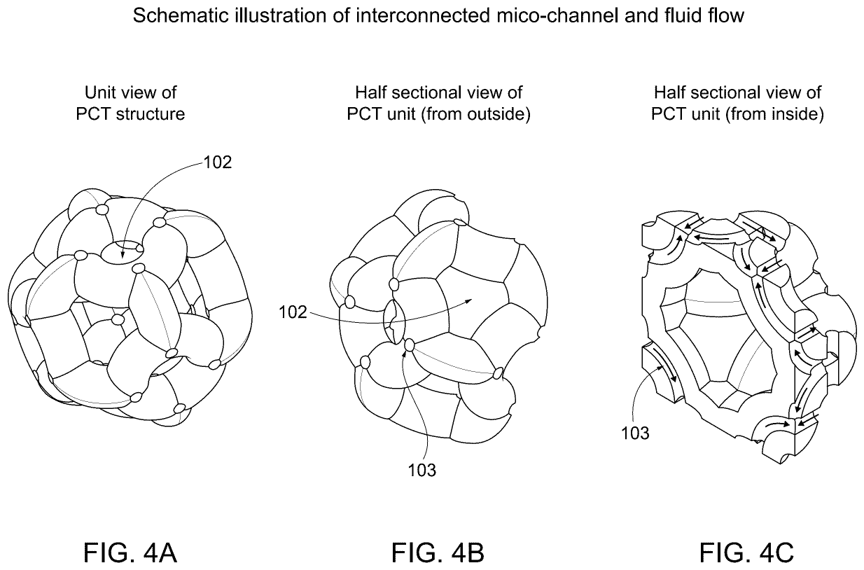

Referring now to FIG. 4, an exemplary PCT is schematically illustrated therein. More particularly, FIG. 4A schematically depicts a unit view of an overall structure of a PCT according to an embodiment of the present disclosure, while FIG. 4B depicts a half sectional view of the PCT from outside. FIG. 4C also schematically depicts a half sectional view of the PCT from inside, schematically illustrating fluid flow through the secondary micro-channels 103.

In some embodiments, a PCT includes primary macro-pores 102 defined by a plurality of struts 105 (see, e.g., FIGS. 2, 3A and 3B), secondary micro-channels 103 formed in the struts 105, and tertiary sub-micro holes 104 formed in the surfaces of the struts 105. The primary macro-pores 102, which are interconnected with one another, are about 100-600 μm in diameter and are formed throughout the PCT for providing spaces for cells to migrate deep into the PCT and to grow throughout the PCT. The secondary micro-channels 103, which (or at least some of which) are interconnected with one another, are about 20-70 μm in diameter and are formed for providing continuous fluid flow to supply oxygen and nutrients to the cells (as illustrated by the arrows in FIG. 4C) and additional surface areas for cell attachment. The tertiary sub-micro holes 104 are about 80-400 nm in diameter and are formed in the surfaces of the PCT, such as the surfaces of the struts 105, to encourage cells to anchor.

In some embodiments, the PCT is substantially cylindrical (see, e.g., FIGS. 1A-1C). In some other embodiments, the PCT has a diameter about 5-10 mm and/or height about 5-10 mm. The PCT may be provided with other diameters, heights, sizes, dimensions, and/or shapes, depending on application needs and requirements.

In some embodiments, the PCT is made from suitable ceramic materials, including but not limited to, alumina, zirconia, titania, glass oxide, calcium phosphate-based oxide such as hydroxyapatite, tricalcium phosphate, and/or magnesium/strontium/zinc-substituted calcium phosphate-based oxide.

In some embodiments, the PCT includes a plurality of primary cells and/or a plurality of secondary cells seeded thereon or therein. To create a co-culture model, the primary cells include normal cells, such as osteoblast precursors, osteoblasts, osteoclasts, fibroblasts, muscle cells, bone marrow cells, and/or mesenchymal stem cells. In other embodiments, the primary cells may include other normal cells. In some embodiments, the secondary cells include tumor or cancer cells, such as osteosarcoma, chondrosarcoma, Ewing's sarcoma, fibrosarcoma, and/or breast cancer cell. In other embodiments, the secondary cells may include other tumor or cancel cells. In some embodiments, the primary cells are introduced into the porous ceramic template to establish a normal cellular microenvironment. After establishing a normal cellular microenvironment, the secondary cancer cells are introduced into the porous ceramic template to mimic an in-vivo 3D cancer model.

In some embodiments, a bioreactor is provided. The bioreactor includes a column connected with a peristaltic perfusion pump operable to circulate a culture medium. In some embodiments, the culture medium comprises a pharmaceutical chemotherapeutic agent and/or another therapeutic agent.

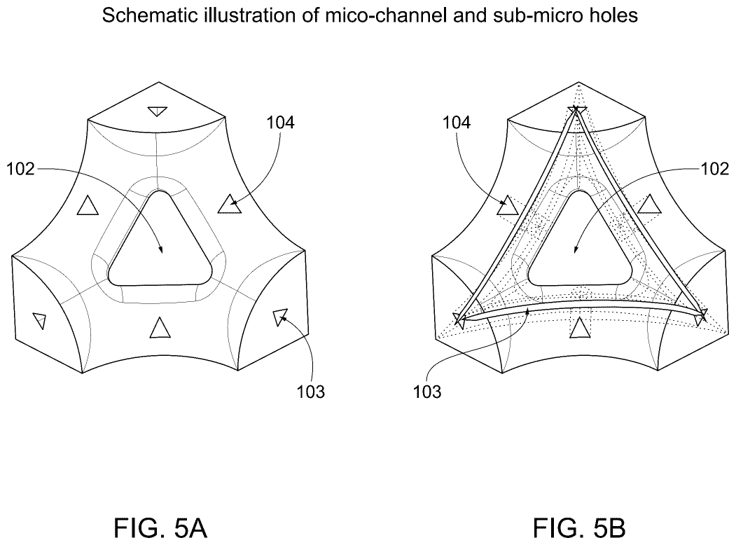

Referring now to FIG. 5, a part of a PCT is schematically illustrated therein, showing secondary micro-channels 103 and tertiary sub-micro holes 104, in accordance with an embodiment of the present disclosure. More particularly, FIG. 5A schematically illustrates a zoomed-in view of an outer structure of the PCT illustrated in FIG. 4B, while FIG. 5B schematically illustrates an internal structure that includes interconnected micro-channels 103 and sub-micro holes 104.

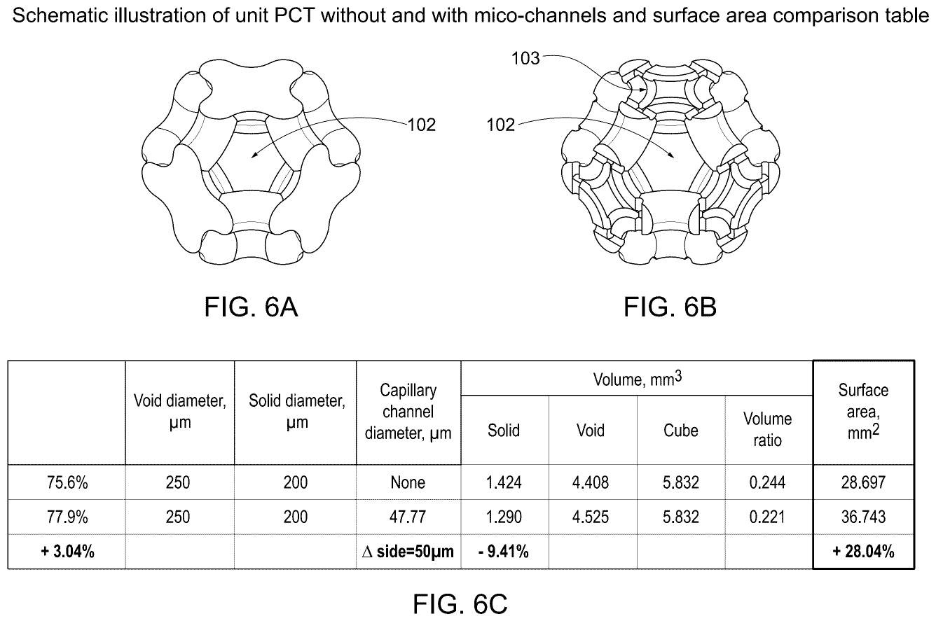

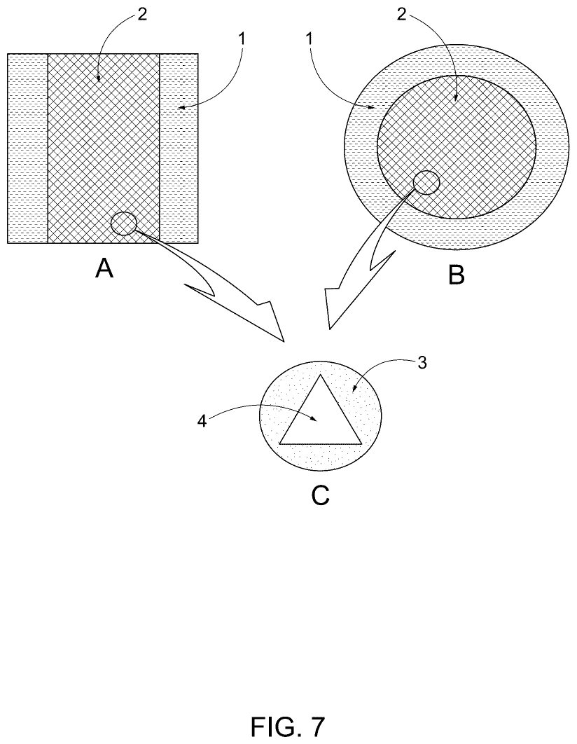

Referring now to FIG. 6, an exemplary schematic illustration of a unit PCT without and with micro-channels 103. FIG. 6A depicts a schematic view of a PCT structure without secondary micro-channels, while FIG. 6B depicts a schematic view of a PCT structure with secondary micro-channels 103. The table in FIG. 6C shows a comparison between the surface areas of the PCT structures without and with micro-channels 103. The overall surface areas were determined using the following method, as disclosed in Oh et al., “Bone marrow absorption and retention properties of engineered scaffolds with micro-channels and nano-pores for tissue engineering: A proof of concept”, Ceramic International, Vol. 39, Issue 7, pages 8401-8410 (2013). A CAD model of the PCTs was developed to investigate the effect micro-channels have on scaffold porosity and surface area per volumetric unit (Solidworkss, Dassault Systems Solidworks, MA, USA). A CAD model of a closed PCT structure (without micro-channels) was developed based on an open-unit truncated octahedron scaffold design, and then 50 μm diameters of the channels inside the curved-rods were created along the axis of the six curved-rods. This unit model was chosen to suitably represent the original 3D structure of the PCTs. This model was easy to create a periodic unit that could be spatially organized to maximize true structural volume of the PCT. To calculate the surface area and porosity of the of the PCTs, the geometric parameters of the model were chosen as follows: 100 μm diameter of the trabecular septum, 500 μm height of the scaffold model unit, and 50 μm diameters of the channels inside the trabecular septum. The porosity and the surface area of the PCTs with and without micro-channels were measured using the functions of the solid and void volumes, and the surface area in the CAD software.

In this comparison, the overall surface area was increased substantially (or by about 142%) in the PCT with secondary micro-channels 103 that are about 20-about 70 μm in diameter, compared to the PCT without secondary micro-channels. On the other hand, the overall porosity was increased only by about 109% with the micro-channels design. In some embodiments, a PCT having an increased overall surface area facilitates cell proliferation in same.

PCTs suitable for use according to the present disclosure generally exhibit biocompatibility, have closely matched mechanical properties when compared to native bone, and possess a mechanism to allow diffusion and/or transport of ions, nutrients, and wastes. The architecture of the PCT (pore size, porosity, interconnectivity, and permeability suitable for ion and transport/diffusion of nutrients and wastes) allows sustained cell proliferation and differentiation within the PCT.

In some embodiments, the PCT of the present disclosure is a single-density or multi-density porous structure that promotes cellular and/or nutrient infiltration. In some embodiments where the PCT possesses interconnected primary macro-pores, secondary micro-channels, and sub-micro holes, all or only a portion of the PCT may possess the micro-channels and/or sub-micro holes. In some embodiments, the micro-channels connect to sub-micro holes, while in some other embodiments they do not. In some embodiments, the macro-pores, micro-channels, and sub-micro holes are of uniform shape, while in some other embodiments they are distinctly shaped. In some embodiments, the macro-pores, micro-channels, and sub-micro holes are of uniform size, while in other embodiments they are of a variety of sizes. In some embodiments, the PCT includes latent pores that become actual pores after the PCT is placed in a perfusion bioreactor as described herein.

The PCT may be composed on a single type of material, or more than one material, or composite of materials such as one of calcium phosphate-based oxides or mixture with other ceramic materials. Various methods known in the art may be used for fabrication of a PCT suitable for use according to embodiments of the present disclosure. These include, without limitation, leaching processes, gas foaming processing, supercritical carbon dioxide processing, sintering, phase transformation, freeze-drying, cross-linking, molding, porogen melting, polymerization, melt-blowing, and salt fusion. In some embodiments, the PCT of the present disclosure is made in accordance with the processes disclosed in one of the following documents: (1) Oh et al., “Bone marrow absorption and retention properties of engineered scaffolds with micro-channels and nano-pores for tissue engineering: A proof of concept”, Ceramics International, Vol. 39, Issue 7, pages 8401-8410 (2013); (2) Oh et al., “Effect of capillary action on bone regeneration in micro-channeled ceramic scaffolds”, Ceramics International, Vol. 40, Issue 7, Pages 9538-9589 (2014); (3) Hong et al., “Capillary action: enrichment of retention and habitation of cells via micro-channeled scaffolds for massive bone defect regeneration”, Journal of Materials Science: Materials in Medicine, Vol. 25, Issue 8, Pages 1991-2001 (2014); (4) Oh et al., “Distinctive Capillary Action by Micro-channels in Bone-like Templates can Enhance Recruitment of Cells for Restoration of Large Bony Defect”, J Vis. Exp. (103), e52947, doi: 10.3791/52947 (2015), http://www.jove.com/video/52947; and (5) U.S. Pat. No. 8,916,228 entitled “Bi-layered bone line scaffolds” and issued Dec. 23, 2014.

Discussed below are various non-limiting examples of PCT components and methods for fabricating the PCT according to embodiments of the invention.

### A. Scaffold (PCT) Components

The scaffolds of the present invention may be composed of a variety of components. The components can be obtained from natural sources, commercial sources, or can be chemically synthesized. In particular embodiments, the scaffold includes a calcium phosphate. Regarding natural sources, calcium phosphates are found in bone, teeth and shells of a large variety of animals. It exists in a variety of forms known in the art, and non-limiting examples include hydroxyapatite (Hydroxyapatite, Ca10(PO4)6(OH)2, Ca/P=1.67), tricalcium phosphate (TCP, Ca3(PO4)2, Ca/P=1.5)) and brushite (CaHPO4·2H20, Ca/P=1. Hydroxyapatite has characteristics similar to mineralized matrix of natural bone, and is biocompatible. Non-limiting examples of calcium compounds include calcium nitrate tetrahydrate, calcium nitrate, and calcium chloride. Non-limiting examples of phosphorus compounds include triethylphosphate, sodium phosphate, and ammonium phosphate dibasic. One of ordinary skill in the art would be familiar with the wide variety of calcium phosphates known in the art, and sources of such compounds.

There are several reported methods for the synthesis of hydroxyapatite. Processes include aqueous colloidal precipitation, sol-gel, solid-state and mechano-chemical methods. Information regarding stabilized calcium phosphate complexes can be found in U.S. Patent App. Pub. No. 20080075675. Additional information regarding synthesis of hydroxyapatite can be found in U.S. Patent App. Pub. No. 20080095820 and U.S. Pat. No. 6,171,610.

One method includes reacting calcium and a non-acidic ionic phosphate, such as trisodium phosphate, in the presence of hydroxyl ions. U.S. Pat. Nos. 5,258,044, 5,306,305, 5,543,019, 5,650,176, 5,676,976, 5,683,461, 5,783,217, 5,843,289, 6,027,742, 6,033,582, 6,117,456, 6,132,463 and 6,214,368 disclose methods of synthesizing calcium phosphate particles and a variety of biomedical uses.

The scaffolds of the present invention may include any component known to those of ordinary skill in the art to be suitable for inclusion in a biomedical scaffold. Other non-limiting examples of such components include polymethylmethacrylate (PMMA), calcium sulfate compounds, calcium aluminate compounds, aluminum silicate compounds, bioceramic materials, or polymers. Examples of the bioceramic material include calcium phosphate-based oxide, such as apatite, BIOGLASS™, glass oxide, titania, zirconia, and alumina. Other suitable materials include alginate, chitosan, coral, agarose, fibrin, collagen, bone, silicone, cartilage, aragonite, dahlite, calcite, amorphous calcium carbonate, vaterite, weddellite, whewellite, struvite, urate, ferrihydrite, francolite, monohydrocalcite, magnetite, goethite, dentin, calcium carbonate, calcium sulfate, calcium phosphosilicate, sodium phosphate, calcium aluminate, a-tricalcium phosphate, a dicalcium phosphate, β-tricalcium phosphate, tetracalcium phosphate, octacalcium phosphate (OCP), fluoroapatite, chloroapatite, magnesium-substituted tricalcium phosphate, carbonate hydroxyapatite, and combinations and derivative thereof. Examples of silicon compounds include tctracthylorthosilicate, 3-mercaptopropyltrimethoxysilane, and 5,6-epoxyhexyltriethoxysilane.

The scaffolds of the present invention may optionally include any number of additional additives. In some embodiments, additives are added to a portion of the scaffold. For example, a scaffold may include additives in the cortical shell but not in the inner trabecular core, or vice versa. In some embodiments, there are additives in both the cortical shell and trabecular core. Non-limiting examples of additives include radiocontrast media to aid in visualizing the scaffold with imaging equipment. Examples of radiocontrast materials include barium sulfate, tungsten, tantalum, or titanium. Additives that include osteoinductive materials may be added to promote bone growth into the hardened bone augmentation material. Suitable osteoinductive materials may include proteins from transforming growth factor (TGF) beta superfamily, or bone-morphogenic proteins, such as BMP2 or BMP7.

In preferred embodiments of the present invention the scaffolds set forth herein are biocompatible. The term “biocompatible” is intended to describe any material which upon implantation does not elicit a substantial detrimental response in vivo.

In particular embodiments of the present invention, the scaffold is biodegradable, bioerodible, or resorbable, unless a permanent matrix is desired. The terms “biodegradable”, “bioerodable” and “resorbable” arc used herein interchangeably. When used to characterize materials, they refer to materials that degrade under physiological conditions to form a product that can be metabolized or excreted without damage to the subject. In certain embodiments, the product is metabolized or excreted without permanent damage to the subject. Biodegradable materials may be hydrolytically degradable, may require cellular and/or enzymatic action to fully degrade, or both. Other degradation mechanisms, e.g., thermal degradation due to body heat, are also envisioned. Biodegradable materials also include materials that are broken down within cells. Degradation may occur by hydrolysis, enzymatic processes, phagocytosis, or other processes.

Either natural or synthetic polymers can be used to form the scaffold matrix. U.S. Pat. Nos. 6,171,610, 6,309,635 and 6,348,069 disclose a variety of matrices for use in tissue engineering.

In some embodiments which include an outer cortex and an inner core, only the outer cortex is biodegradable. In further embodiments, only the inner core is biodegradable. Non-limiting examples of synthetic polymers suitable for inclusion in the scaffolds of the present invention include fibrin, collagen, glycosaminoglycans (GAGs), such as chitin, chitosan and hyaluronic acid, polysaccharides, such as starch, carrageenan, alginate, heparin, glycogen and cellulose, polylactide (PLA), polylactide-co-glycolide (PLGA), polyglycolic acid (PGA), polyurethanes, polycaprolactone, polymethyl methacrylate (PMMA), polyamino acids, such as poly-L-lysine, polyethyleneimine, poly-anhydrides, polypropylene-fumarate, polycarbonates, polyamides, polyanhydrides, polyortho esters, polyacetals, polycyanoacrylates and degradable polyurethanes.

Useful non-erodible polymers include without limitation, polyacrylates, ethylene-vinyl acetate polymers and other acyl substituted cellulose acetates and derivatives thereof, non-erodible polyurethanes, polystyrenes, polyvinyl chloride, polyvinyl fluoride, poly(vinyl imidazole), chlorosulphonated polyolifins, polyethylene oxide, polyvinyl alcohol, TEFLON™, nylon, stainless steel, cobalt chrome, titanium and titanium alloys, and bioinert ceramic particles (e.g., alumina and zirconia particles), polyethylene, polyvinylacetate, polymethylmethacrylate, silicone, polyethylene oxide, polyethylene glycol, polyurethanes, and natural biopolymers (e.g., cellulose particles, chitin, keratin, silk, and collagen particles), and fluorinated polymers and copolymers (e.g., polyvinylidene fluoride).

In some embodiments, the scaffold is coated with compounds to facilitate attachment of cells to the scaffold. Examples of such compounds include basement membrane components, agar, agarosc, gelatin, gum arabic, collagen types I, II, III, IV, and V, fibronectin, laminin, glycosaminoglycans, polyvinyl alcohol, and mixtures thereof.

In some embodiments, mammalian cells are incorporated into the scaffolds. For example, mammalian cells may be seeded or cultured with the scaffolds of the present invention prior to implantation in a subject. Examples of such cells include, but are not limited to, bone marrow cells, smooth muscle cells, stromal cells, stem cells, mesenchymal stem cells, synovial derived stem cells, embryonic stem cells, umbilical cord blood cells, umbilical Wharton's jelly cells, blood vessel cells, chondrocytes, osteoblasts, osteoclasts, precursor cells derived from adipose tissue, bone marrow derived progenitor cells, kidney cells, intestinal cells, islets, beta cells, pancreatic ductal progenitor cells, Sertoli cells, peripheral blood progenitor cells, fibroblasts, glomus cells, keratinocytes, nucleus pulposus cells, annulus fibrosus cells, fibrochondrocytes, stem cells isolated from adult tissue, oval cells, neuronal stem cells, glial cells, macrophages and genetically transformed cells or combination of the above cells. The cells can be seeded on the scaffolds for a short period of time just prior to implantation (such as one hour, six hours, 24 hours), or cultured for longer periods of time (such as 2 days, 3 days, 5 days, 1 week, 2 weeks) to promote cell proliferation and attachment within the scaffold prior to implantation.

### B. Fabrication of Scaffolds (PCTs)

### 1. Formation of Pores and Micro-Channels

Formation of pores and micro-channels in the scaffolds set forth herein may be accomplished using any method known to those of ordinary skill in the art. In some embodiments, as discussed in the Example section below, pores and micro-channels are created in a scaffold using a template, such as a sponge. A composition, such as a calcium phosphate, is then applied to the template. For example, in some embodiments the method includes (a) contacting a porous polymer sponge with a composition that includes a suitable material for scaffold formation, wherein at least a portion of the sponge becomes coated with the composition; and (b) drying the composition-coated sponge, wherein a bone scaffold is formed. In some embodiments, the sponge is burned out of the scaffold.

Other methods of creating pores or micro-channels that may be applied in the context of the present invention include, but are not limited to, leaching processes, gas foaming processing, supercritical carbon dioxide processing, sintering, phase transformation, freeze-drying, cross-linking, molding, porogen melting, polymerization, melt-blowing, and salt fusion (reviewed in Murphy et al., 2002; Karageorgiou et al., 2005). Porosity may be a feature of the composition during manufacture or before implantation, or the porosity may only be available after implantation. Additional information regarding formation of pores in a scaffold can be found in U.S. Patent App. Pub. No. 20080069852. In some embodiments, micro-channels and/or larger channels are drilled into the scaffold following molding.

The present invention also contemplates applications using porogens to create latent pores in a composite. These latent pores may arise from including porogens in the composite. The porogen may be any chemical compound that will reserve a space within the composite while the composite is being molded and will diffuse, dissolve, and/or degrade prior to or after implantation leaving a pore in the composite. Porogens may be of any shape or size, such as spheroidal, cuboidal, rectangular, elongated, tubular, fibrous, disc-shaped, platelet-shaped, or polygonal. In certain embodiments, the porogen is granular. The porogen may be a gas, liquid, or solid. Exemplary gases that may act as porogens include carbon dioxide, nitrogen, argon, or air. Exemplary liquids include water, organic solvents, or biological fluids (such as blood, lymph, plasma). Examples of possible solid porogens include water soluble compounds such as carbohydrates (e.g., sorbitol, dextran poly(dextrose), starch), salts, sugar alcohols, natural polymers, synthetic polymers, and small molecules.

Additional information regarding incorporation of pores into a material can be found in U.S. Patent App. Pub. No. 20080103227.

### 2. Shaping

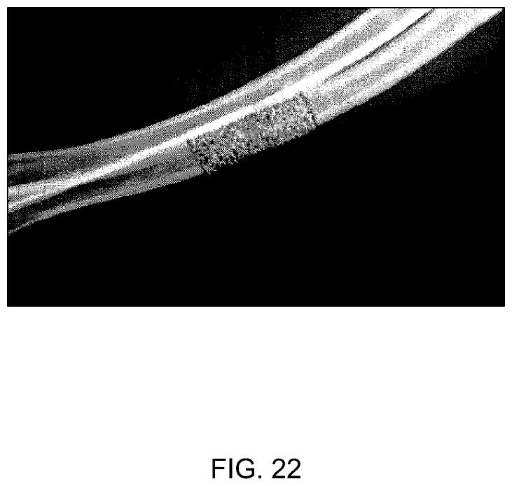

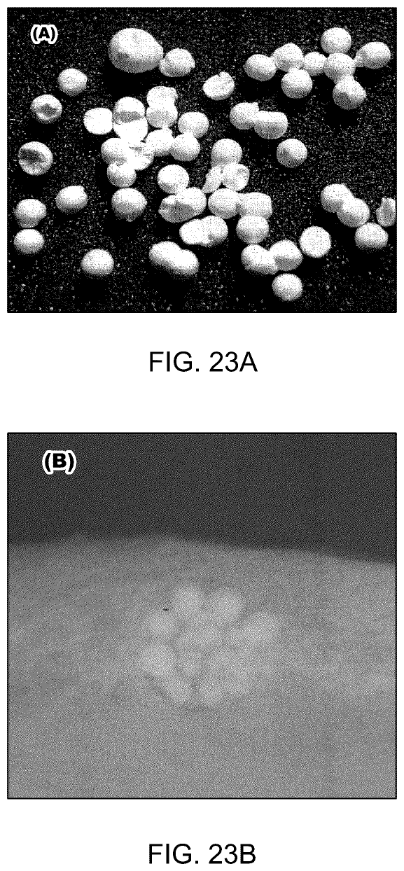

The scaffolds set forth herein can be formed into a desired shape using any method known to those of ordinary skill in the art. For example, the scaffold may be molded into a desired shape or fractured into granules. The granules retain the essential pores and/or micro-channels. The scaffold may be configured by the surgeon prior to implantation or at the time of implantation into a desired shape, such as a curved custom-made scaffold to fit the shape, anatomical structure and size of tibia shown as FIG. 22. In some embodiments, a scaffold of the present invention is fractured into granules which in turn can be packed into a bony defect by the surgeon. The granules may be of a uniform size, or of varying sizes.

### 3. Formation of Cortex and Coatings

Certain embodiments of the present scaffolds include an outer cortex or coating. Formation of an outer cortex or coating on a core component can be performed using any method known to those of ordinary skill in the art. As discussed in the Examples below, a template (such as a sponge) may be applied in forming an outer cortex on a scaffold. U.S. Patent App. Pub. No. 20080097618 provides information regarding deposition of calcium phosphate coatings on surfaces. In some embodiments, forming a coating involves dipping or immersing a scaffold in a composition or a plasma spray deposition process. Information concerning immersion techniques can be found in U.S. Pat. Nos. 6,143,948, 6,136,369 and 6,344,061.

### C. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Procedure for Porous Calcium Phosphate Scaffold (PCT) Fabrication

### 1.1 Polymeric Sponge Template Preparation

Selection of the template material: A polyurethane (PU) sponge template is used to produce uniform interconnected porous calcium phosphate scaffolds. This sponge is used to provide the primary structure for the formation of the scaffold struts as well as the formation of secondary micro-channels within the scaffold struts. The polyurethane sponge template chosen may range from 45 pores per inch (ppi) to 80 ppi for the inner trabecular core, depending on the final desired pore size. The pore sizes in the inner trabecular core may range from 150 μm to 800 μm after sintering to allow bone cell migration, blood vessel vascularization, and nutrient supply. Additionally, the 80 ppi to 100 ppi polyurethane sponge template or solid calcium phosphate ceramics (with channels and/or pores produced by slip casting) may be chosen for the outer cortical shell, depending on the final desired pore size. The pores and/or channel or holes for the outer cortical shell may be in the range of about 50 μm to about 250 μm after sintering, depending on the desired application place.

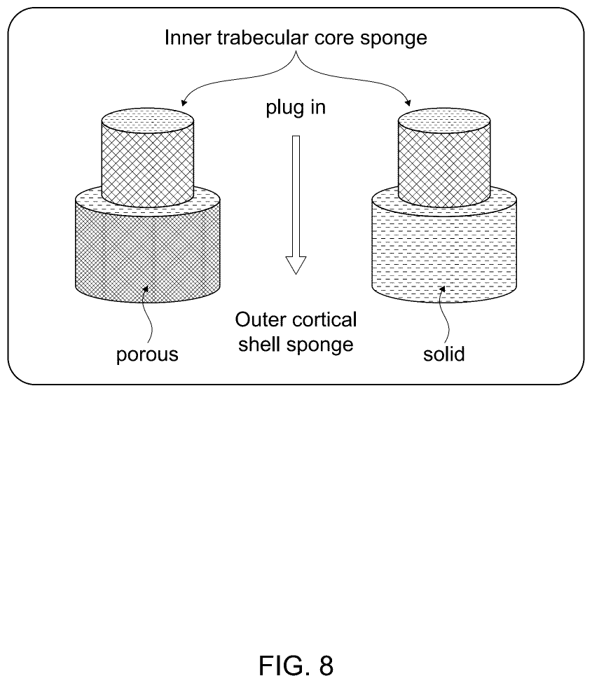

Template sponge preparation: The polyurethane sponge is used as a template and is first cut to the desired shape and dimension. The cut polyurethane is then ultrasonically treated in 10% to 15% sodium hydroxide (NaOH) solution for 20 to 30 minutes, followed by cleaning in flowing water for 30 to 60 minutes. The treated polyurethane is then rinsed with distilled water. During cleaning with water and rinsing with distilled water, the polyurethane is squeezed and then allowed to expand for 5 to 10 times in order to remove the residual NaOH inside polyurethane sponge template. The polyurethane sponge template is then ultrasonically cleaned again in distilled water for 20 to 30 minutes. This is followed by squeezing the template sponge with paper towel in order to remove excess water. The template sponge is then placed in an oven at 60° C. to 80° C. until completely drying. The completely dried sponge template for the inner trabecular core is then snuggly fitted into the outer sponge template for the cortical shell or solid outer shell (with channels and/or holes depends on final desired structure and application). At this point, as shown in FIG. 8, the sponge template is now one piece (outer cortical shell and inner trabecular core) and is ready for calcium phosphate coating.

### 1.2 1st Time Coating Calcium Phosphate Slurry Preparation

In order to produce a stable and well-shaped three-dimensional interconnective porous scaffold, a preferred binder is added to the dispersion. The binders used may be carboxymethylcellulose, polyvinyl alcohol, starch, sodium silicate, polyvinyl butyral, methacrylate emulsion, water soluble polyacrylate, polyacrylic acid, polyethylene glygol, etc. In order to avoid slurry agglomeration and cracking of the scaffold during drying, a dispersant and drying agent is added to the dispersion. The preferred binders are polyvinyl alcohol, carboxymethylcellulose and sodium silicate. In this invention, ammonium polyacrylate and N, N-dimethylformamide will be use as a dispersant and a drying agent, respectively. The preferred amount of polyvinyl alcohol, carboxymethylcellulose, sodium silicate, and methacrylate emulsion added are 2% to 4% by mass, 2% to 4% by mass, 1% to 2% by mass and 1%-2% by mass, respectively (based on 100% by mass of calcium phosphate powder).

The polyvinyl alcohol is added to distilled water, heated and stirred until the polyvinyl alcohol is completely dissolved. The solution should be clear after complete dissolution of the polyvinyl alcohol. As the solution is cooled down to room temperature, carboxymethylcellulose is added. After complete dissolution of the carboxymethylcellulose, sodium silicate solution and methacrylate emulsion are added to the mixture and stirred. Additionally, 5% to 7% by mass of ammonium polyacrylate dispersant and 3% to 5% by mass of N, N-dimethylformamide drying agent are added to the mixture and stirred continuously. The calcium phosphate powders are then slowly dispersed into the solution, followed by stirring. In this invention, calcium phosphate powder is generic and refers to all the different phases of the calcium phosphate group, including hydroxyapatite, tricalcium phosphate, amorphous calcium phosphate, monocalcium phosphate, dicalcium phosphate, octacalcium phosphate, tetracalcium phosphate, fluorapatite, carbonated apatite and the different mixtures of the different phases. Using continuously slow heating, the solution is slowly stirred in order to evaporate the water content and until a powder/liquid ratio of 1.20 to 1.50 is obtained. The slurry is then allowed to cool down to the room temperature before being used for coating.

### 1.3 First Time Calcium Phosphate Coating, Drying and Sintering

The treated one-piece sponge template containing the outer cortical shell and inner trabecular core (from section 1.1) is then immersed into the calcium phosphate slurry until the calcium phosphate slurry is fully absorbed into the sponge template scaffold. The polyurethane is then rolled on a glass plate with rod bar then allowed to expand for 5 to 10 times in order to remove excess slurry. After removing the excess slurry, some of pores may be clogged up with slurry because of high slurry viscosity. In order to ensure interconnectivity, uniformity, and open pores, the scaffolds are slightly blown with air. In this process, it is preferred that the template is homogeneously coated on the inside and outer the sponge template. If this homogeneous coating is not achieved, the calcium phosphate-coated sponge template scaffold will collapse after sintering or fracture during handling. Additionally, the homogeneous coating is preferred for the successful production of the secondary micro-channels within the main scaffold struts.

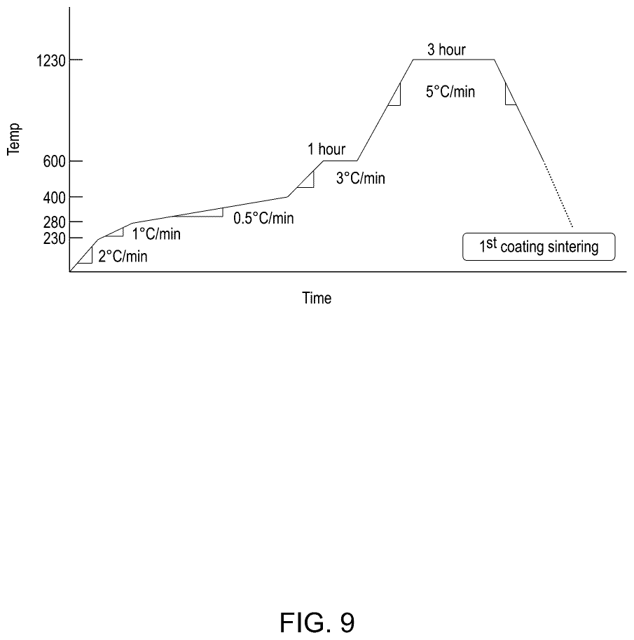

Based on the thermoanalysis of polyurethane sponge template and nano-size powders, the calcium phosphate-coated sponge template scaffolds dry at 25° C. to 35° C. and at 60%-80% humid environment. Drying time will range from 12 to 72 hours, depending on the size of the sponge template scaffolds. After drying, the calcium phosphate-coated sponge template scaffold typically shrinks about 8% to 10%. After the sponges are completely dried, the coated sponges are then placed on an alumina plate, placed in a high temperature furnace, and sintered for 2 to 5 hours at 1200° C. to 1250° C. using an 8-step sintering profile shown in FIG. 9. Sintering will further shrink the calcium phosphate-coated sponge template scaffolds by 22%-25%.

### 1.4 Second Time Coating Calcium Phosphate Suspension Preparation

The second time coating is performed to fill up coating defects from first time coating performed in section 1.3. This second time coating will improve the compressive strength of the scaffold and to ensure a more rounded strut to enhance cell attachment. In order to make the second time coating calcium phosphate suspension, different amounts of the same binders and chemical agents used in the first time coating calcium phosphate slurry preparation (section 1.2) are utilized. However, the concentrations of the binders and chemical agents used are different from the 1st time coating calcium phosphate slurry preparation (section 1.2). The preferred amount of polyvinyl alcohol, carboxymethylcellulose, sodium silicate, and methacrylate emulsion used in the second time coating calcium phosphate suspension preparation are about 3% to about 7% by mass, about 3% to about 7% by mass, about 1% to about 2% by mass and about 1% to about 2% by mass, respectively (based on 100% by mass of calcium phosphate powder).

The polyvinyl alcohol is added to distilled water, heated and stirred until the polyvinyl alcohol is completely dissolved. The solution should be clear after complete dissolution of the polyvinyl alcohol. As the solution is cooled down to room temperature, carboxymethylcellulose is added. After complete dissolution of the carboxymethylcellulose, sodium silicate solution and methacrylate emulsion are added to the mixture and stirred. Additionally, 7% to 10% by mass of ammonium polyacrylate dispersant and 5% to 7% by mass of N, N-dimethylformamide drying agent are added to the mixture and stirred continuously. The calcium phosphate powders are then slowly dispersed into the solution, followed by stirring. Using continuously slow heating, the solution is slowly stirred in order to evaporate the water content and until a powder/liquid ratio of 0.4 to 0.5 is obtained. The slurry is then allowed to cool down to the room temperature before being used for coating.

### 1.5 Second Coating, Drying and Sintering

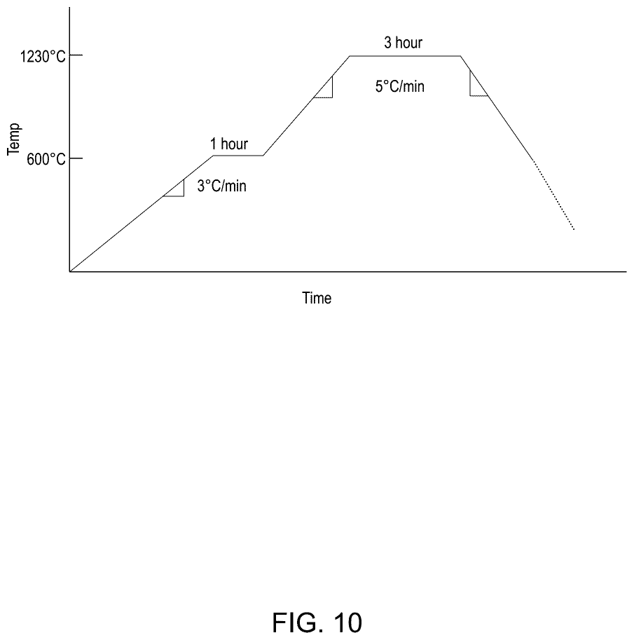

Scaffolds after the first coating and sintering (section 1.3) is immersed into second time coating calcium phosphate suspension (section 1.4) for 10 to 20 seconds. After immersion, the scaffolds arc removed from the suspension. Most of the scaffold pores will be clogged up by the calcium phosphate suspension. In order to ensure interconnectivity, uniformity, and open pores, the scaffolds are slightly blown with air. The calcium phosphate-coated sponge template scaffolds are then dried at 25° C. to 35° C. and at 60% to 70% humid environment. Drying time will range from 12 to 48 hours, depending on the size of the sponge template scaffolds. After the sponges are completely dried, the coated sponges are then placed on an alumina plate, placed in a high temperature furnace, and sintered for 2 to 3 hours at 1200° C. to 1250° C. using a 5-step sintering profile shown in FIG. 10.

1.6 Solid Outer Shell with Channels and/or Holes Calcium Phosphate Suspension Preparation

For scaffolds to be used in loading bearing applications, solid outer shells with channels having diameters ranging from about 100 μm to about 200 μm and/or holes having diameter ranging from about 200 μm to about 500 μm can fabricated by slip casting and freeze-drying method. In order to produce a slip casting suspension, the same binders and same chemical agents in section 1.2 as well as section 1.4 are used. The preferred amount of polyvinyl alcohol, carboxymethylcellulose, sodium silicate, and methacrylate emulsion used in the second time coating calcium phosphate suspension preparation are 3% to 7% by mass, 3% to 7% by mass, 0.5% to 1% by mass and 0.5% to 1% by mass, respectively (based on 100% by mass of calcium phosphate powder).

The polyvinyl alcohol is added to distilled water, heated and stirred until the polyvinyl alcohol is completely dissolved. The solution should be clear after complete dissolution of the polyvinyl alcohol. As the solution is cooled down to room temperature, carboxymethylcellulose is added. After complete dissolution of the carboxymethylcellulose, sodium silicate solution and methacrylate emulsion are added to the mixture and stirred. Additionally, about 7% to about 10% by mass of ammonium polyacrylate dispersant and about 5% to about 7% by mass of N, N-dimethylformamide drying agent are added to the mixture and stirred continuously. The calcium phosphate powders are then slowly dispersed into the solution, followed by stirring. Continuously slow heat and stir the solution to evaporate the water content until a powder/liquid ratio of about 0.4 to about 0.5 is obtained. The slurry is then allowed to cool down to the room temperature before being used for slip casting.

### 1.7 Slip Casting, Freeze Dry and Sintering for Solid Outer Shell

The prepared slip casting calcium phosphate suspension is poured into a designed gypsum mold with a polyurethane sponge mesh having about 10 ppi to about 20 ppi. The gypsum mold containing polyurethane sponge is then rolled at 10 to 20 rpm until all water is completely absorbed by the gypsum mold and a desired thickness is achieved. The gypsum mold is then dried using a freeze dryer for a period ranging from 24 to 72 hours. After drying, the solid outer shell is separated from gypsum mold and holes with diameter ranging from 200 μm to 500 μm are drilled through the outer shell. The well-prepared outer shell is then placed in the high temperature furnace and sintered using a 8-step sintering profile shown in FIG. 9. After sintering, the about 10 ppi to about 20 ppi polyurethane sponge mesh is burnt out, resulting in the formation of channels within the shell.

### 1.8 Antibacterial Calcium Phosphate Sol Preparation

Variable calcium phosphates ceramics such as hydroxyapatite (Ca/P=1.67), tri-calcium phosphate (Ca/P=1.50), meta-calcium phosphate (Ca/P=0.50), calcium polyphosphate (Ca/P=0.50), dicalcium phosphate dehydrate (Ca/P=1.00), monocalcium phosphate anhydrous (Ca/P=0.50) sol can be synthesized using the correct amount of calcium and phosphorous precursors and with controlled aging conditions. Additionally, antibacterial calcium phosphate sol can be synthesized using silver- or zinc-doped in the phosphorous precursor. To make the calcium precursor, a correct amount of calcium nitrate tetrahydrate [Ca(NO3)2·4H2O (Aldrich 99%, USA)] is fluxed in sufficient amount of methyl alcohol and is dehydrated at temperatures ranging from 150° C. to 200° C. After solvent evaporation, the calcium precursor is refluxed in sufficient amount of methyl alcohol. In order to make the phosphorous precursor, a correct amount of triethyl phosphite [(C2H5O)3P (Fluka 97%, Japan)] is fluxed in sufficient amount of methyl alcohol. This fluxed phosphorous precursor is also pre-hydrolyzed for 5 hours in the presence of a catalyst (acetic acid [CH3COOH] containing 0.5 mol % to 1.5 mol % of silver nitrate [Ag(NO3)] or 0.5 mol % to 1.5 mol % of zinc nitrate hydrate [Zn(NO3)2·xH2O] and distilled water [H2O]). The silver- or zinc-doped calcium phosphate sol is then synthesized by reacting calcium and phosphorus precursors for a period of 1 to 2 hours and with vigorous stirring. The reaction is performed under an argon atmosphere. The synthesized silver- or zinc-doped calcium phosphate sol is then filtrated through a 0.20 μm to 0.45 μm syringe filter, followed by aging at temperatures ranging from 40° C. to 80° C. and for a period ranging from 12 to 204 hours. After aging, the calcium phosphate sol viscosity will be between about 8.0 cps to about 160 cps, depending on the aging temperature, aging time, methods of sealing the beakers/vials containing the precursor during aging, and whether aging is performed in air circulation or without circulation condition. This means the calcium phosphate sol viscosity will govern the thickness, porosity, and density of the coating layer.

### 1.9 Calcium Phosphate Sol Coating, Drying and Sintering

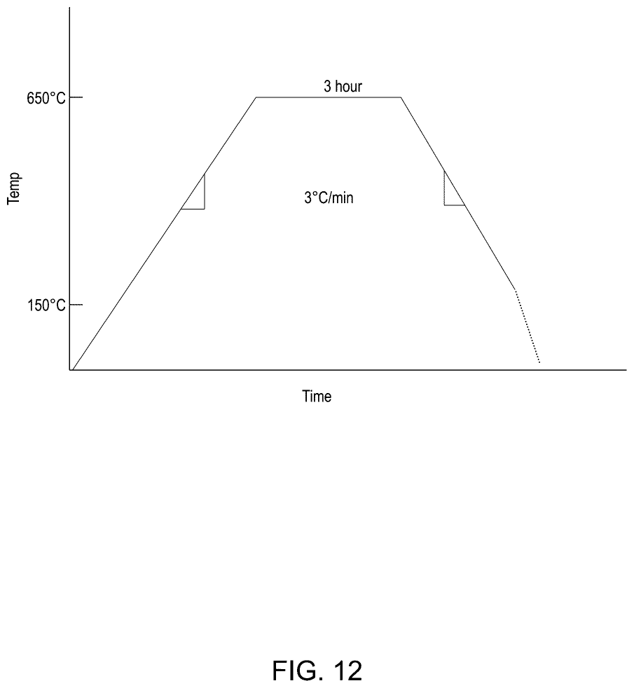

The fabricated porous calcium phosphate scaffolds from section 1.5 and 1.7 are immersed in the aged calcium phosphate sol doped with or without silver or zinc. After immersing for 5 to 10 seconds, the scaffolds are removed from the sol and centrifuged to remove excess sol. The calcium phosphate sol coated scaffold is then baked and dried in an oven at temperatures ranging from 50° C. to 100° C. and for a period ranging from 3 to 8 hours. After completely dried, the calcium phosphate sol-coated scaffolds are then heat-treated at temperatures ranging from 600° C. to 700° C. using a muffle furnace in air for a period ranging from 1 hour to 5 hours shown in FIG. 12.

### Example 2: Examples of Fabricated Hydroxyapatite Scaffolds

Using the process described in Example 1, an example on how this technology is used to fabricate hydroxyapatite scaffolds coated with silver-doped hydroxyapatite is as follows:

### 2.1 Polymetric Sponge Template Preparation for Bi-Layered Porous Scaffold Fabrication:

A 60 ppi (pore per inch) polyurethane sponge template is chosen for trabecular core fabrication and the 100 ppi polyurethane sponge template is chosen for outer cortical shell fabrication. The polyurethane sponge template for the trabecular core is cut to a shape of a solid cylinder with a length of 36 mm and a diameter of 28 mm. The polyurethane sponge template for the outer cortical shell is cut to resemble a cylindrical pipe and is hollow core in the middle. Dimension for the polyurethane sponge template for the outer cortical shell is 36 mm in length, with an outer diameter of 30 mm and an inner diameter of 28 mm, thereby having a 2 mm wall thickness. These polyurethane sponges are ultrasonically treated in 10% sodium hydroxide (NaOH) solution for 20 minutes, followed by cleaning in flowing water for 40 minutes and then rinsed with distilled water. During cleaning, the sponges are squeezed and then allowed to expand for 10 times to remove the residual NaOH inside polyurethane sponge templates. These sponges are then ultrasonically cleaned again in distilled water for 30 minutes. The sponges are then squeezed with paper towels to remove excess water. This is followed by drying at 80° C. in an oven for 5 hours until completely dry is achieved. After completely drying, the polyurethane sponge template for the trabecular core is then snuggly fitted into the pipe-like polyurethane sponge template for the outer cortical shell or pipe-like solid outer shell (with channels and/or holes depends on final desired structure and application). At this point, as shown in FIG. 8, the sponge template is now one piece (outer cortical shell and inner trabecular core).

### 2.2 First Time Coating Hydroxyapatite Powder and β-Tricalcium Phosphate Mixed Slurry Preparation

As an example of this invention, nano-sized hydroxyapatite powder and nano-sized β-tricalcium phosphate powder are used for the fabrication of scaffolds because of their ability to sinter. A 3% (by mass) polyvinyl alcohol (molecular weight of 89,000 to 98,000) is added to 20 ml of distilled water, heated on the hot plate to 60° C. and stirred until the polyvinyl alcohol is completely dissolved. The solution should be clear after complete dissolution of the polyvinyl alcohol. As the solution is cooled down to room temperature, a 3% (by mass) carboxymethylcellulose (molecular weight of 10,000; viscosity of 53,000 cps at 25° C.) is added. After complete dissolution of the carboxymethylcellulose, a 1% (by mass) of sodium silicate solution and a 1% (by mass) of methacrylate emulsion are added to the mixture and stirred. Additionally, a 7% (by mass) of ammonium polyacrylate dispersant and a 5% (by mass) of N, N-dimethylformamide drying agent are added to the mixture and stirred continuously. The percentage of these binders and drying agents are based on 100% by mass of calcium phosphate powder. Three grams of hydroxyapatite powder and 3 grams of β-tricalcium phosphate powders are then slowly dispersed into the solution, followed by stirring. Using continuously slow heat, the solution is slowly stirred in order to evaporate the water content and until a powder/liquid ratio of 1.50 is obtained. The slurry is then cool down to the room temperature before being used for coating the polyurethane sponges.

### 2.3 First Time Calcium Phosphate Coating, Drying and Sintering

The treated one-piece sponge template containing the (outer cortical shell and inner trabecular core (from section 2.1) is immersed in the first time coating slurry (from section 2.2) until the calcium phosphate slurry is fully absorbed in the sponge template scaffold. While in the slurry, the immersed polyurethane sponge template is manually compressed with the aid of a stirrer and allowed to expand for 8 times. The sponge template is then removed from the slurry and excess slurry is removed by the sponge on glass plate with a rod bar. After removal of the excess slurry, some of the pores in the sponge template may be clogged with slurry because of the high slurry viscosity. In order to ensure interconnectivity, uniformity, and open pores, the scaffolds are slightly blown with air. Based on the thermoanalysis of polyurethane sponge templates and nano-size powders, the calcium phosphate slurry-coated sponge template scaffolds are then dried at 27° C. (in 80% humidity in still air environment) for 60 hours. After drying, the calcium phosphate-coated sponge template scaffolds shrink by 8%. After completely drying, the sponge template scaffolds are placed on an alumina plate and sintered in a furnace. The dried calcium phosphate coated sponge template scaffolds are sintered by using the 8-step sintering profile shown in FIG. 9. After sintering, the calcium phosphate coated sponge template scaffolds shrink by 22%. In addition to FIG. 9, details of the 8-step sintering profile, with heating rate and final temperature is as follows:
Step 1: heat 2° C./minute until 230° C.
Step 2: heat 1° C./minute until 280° C.
Step 3: heat 0.5° C./minute until 400° C.
Step 4: heat 3° C./minute until 600° C.
Step 5: keep 600° C. for 1 hour.
Step 6: heat 5° C./minute until 1230° C.
Step 7: keep 1230° C. for 3 hours.
Step 8: cool 5° C./minute to room temperature.

### 2.4 Second Time Coating Calcium Phosphate Suspension Preparation

For the second time coating calcium phosphate suspension, different amounts but the same binders and chemical agents (in section 2.2) are used. A 3% (by mass) polyvinyl alcohol (molecular weight of 89,000 to 98,000) is added to 20 ml of distilled water, heated on a hot plate to 60° C. and stirred until the polyvinyl alcohol is completely dissolved. The solution should be clear after complete dissolution of the polyvinyl alcohol. As the solution is cooled down to room temperature, a 5% (by mass) carboxymethylcellulose (molecular weight of 10,000; viscosity of 53,000 cps at 25° C.) is added. After complete dissolution of the carboxymethylcellulose, a 1% (by mass) of sodium silicate solution and a 1% (by mass) of methacrylate emulsion are added to the mixture and stirred. Additionally, a 10% (by mass) of ammonium polyacrylate dispersant and a 7% (by mass) of N, N-dimethylformamide drying agent are added to the mixture and stirred continuously. The percentage of these binders and drying agents are based on 100% by mass of calcium phosphate powder. 1.5 grams of hydroxyapatite powder and 1.5 grams of β-tricalcium phosphate powders are then slowly dispersed into the solution, followed by stirring. Using continuously slow heat, the solution is slowly stirred in order to evaporate the water content and until a powder/liquid ratio of 0.50 is obtained. The slurry is then cool down to the room temperature before being used for coating.

### 2.5 Second Coating, Drying and Sintering

The scaffolds from the first time coating and sintering (from section 2.3) are immersed in the second time coating slurry (from section 2.4) for 20 seconds. The scaffold is then removed from the slurry. Most of the scaffold pores may be clogged with slurry because of the high slurry viscosity. In order to ensure interconnectivity, uniformity, and open pores, the scaffolds are slightly blown with air. The calcium phosphate-coated scaffolds are then dried at 30° C. (in 70% humidity in still air environment) for 24 hours. After complete drying, the calcium phosphate-coated are placed on an alumina plate and sintered in a furnace. The dried calcium phosphate-coated scaffolds are sintered by using the 5-step sintering profile shown in FIG. 10. In addition to FIG. 10, details of the 5-step sintering profile, with heating rate and final temperature is as follows:
Step 1: heat 3° C./minute until 600° C.
Step 2: keep 600° C. for 1 hour.
Step 3: heat 5° C./minute until 1230° C.
Step 4: keep 1230° C. for 3 hours.
Step 5: cool 5° C./minute to room temperature.
   2.6 Solid Outer Shell with Channels and/or Holes Calcium Phosphate Suspension Preparation

In order to make a slip casting suspension, the same binders and same chemical agents (in sections 2.2 and 2.4) are used. A 3% (by mass) polyvinyl alcohol (molecular weight of 89,000 to 98,000) is added to 20 ml of distilled water, heated on the hot plate to 60° C. and stirred until the polyvinyl alcohol is completely dissolved. The solution should be clear after complete dissolution of the polyvinyl alcohol. As the solution is cooled down to room temperature, a 5% (by mass) carboxymethylcellulose (molecular weight of 10,000; viscosity of 53,000 cps at 25° C.) is added. After complete dissolution of the carboxymethylcellulose, a 0.5% (by mass) of sodium silicate solution and a 0.5% (by mass) of methacrylate emulsion are added to the mixture and stirred. Additionally, a 10% (by mass) of ammonium polyacrylate dispersant and a 7% (by mass) of N, N-dimethylformamide drying agent are added to the mixture and stirred continuously. The percentage of these binders and drying agents are based on 100% by mass of calcium phosphate powder. 1.5 grams of hydroxyapatite powder and 1.5 grams of β-tricalcium phosphate powders are then slowly dispersed into the solution, followed by stirring. Using continuously slow heat, the solution is slowly stirred in order to evaporate the water content and until a powder/liquid ratio of 0.50 is obtained. The slurry is then cool down to the room temperature before being used for slip casting.

### 2.7 Slip Casting, Freeze Dry and Sintering for Solid Outer Shell

The prepared slip casting calcium phosphate suspension is poured into a 33 mm length by 27 mm diameter gypsum mold containing a 10 ppi polyurethane sponge mesh. The mold is rolled at 12 rpm until all water is completely absorbed by the gypsum mold and a desired 2 mm thickness is achieved. The gypsum mold is then dried using a freeze dryer for 48 hours. After drying, the solid outer shell is separated from gypsum mold and drilled holes having diameter of 300 μm. In order to fabricate a bi-layered scaffold, the 1^{st} calcium phosphate-coated trabecular core sponge template (section 2.3) is then snuggly fitted into the dried solid outer shell and dried at 27° C. (in 80% humidity and still air environment) for 60 hours. After complete drying, the bi-layered scaffolds are placed on an alumina plate and sintered in a furnace. The dried calcium phosphate-coated scaffolds are sintered by using the 8-step sintering profile shown in FIG. 9 (section 2.3). Following sintering, the bi-layered coatings are then coated using the 2^{nd} time coating calcium phosphate suspension preparation (section 2.4) and sintered using the 2^{nd} coating, drying and sintering procedure (section 2.5).

### 2.8 Antibacterial Calcium Phosphate Sol Preparation

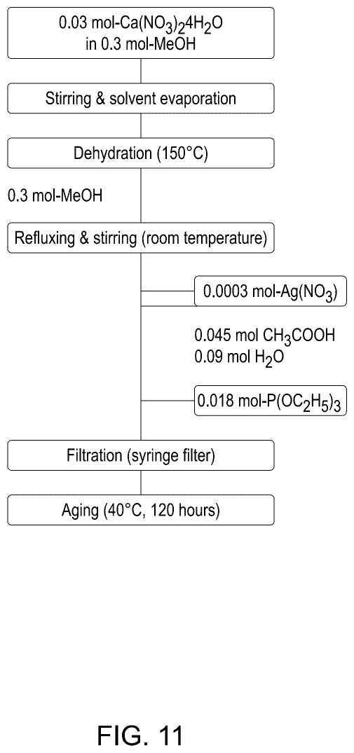

In this invention, silver-doped hydroxyapatite solution is prepared by fluxing 0.03 mol calcium nitrate tetrahydrate [Ca(NO₃)₂·4H₂O (Aldrich 99%, USA)] in 0.3 mol methyl alcohol and is dehydrated at 150° C. After solvent evaporation, the calcium precursor is refluxed in 0.3 mol methyl alcohol for 1 hour. The 0.018 mol triethyl phosphite [(C₂H₅O)₃P (Fluka 97%, Japan)] is fluxed in 0.15 mol methyl alcohol and pre-hydrolyzed for 5 hours in the presence of a catalyst (0.045 mol acetic acid [CH3COOH] with 0.0003 mol silver nitrate [Ag(NO₃)] and 0.09 mol distilled water [H₂O]). The silver-doped hydroxyapatite sol is then synthesized by reacting calcium and phosphorus precursors for 1 hour with vigorous stirring. All work is performed under an argon atmosphere. The synthesized silver-doped hydroxyapatite sol is then filtrated through a 0.45 μm syringe filter and aged at 40° C. for 120 hours. After aging, the viscosity of the silver-doped hydroxyapatite sol viscosity is 36 cps. The flow chart of the silver-doped hydroxyapatite sol preparation is shown in FIG. 11.

### 2.9 Calcium Phosphate Sol Coating, Drying and Sintering

The fabricated porous calcium phosphate scaffold is immersed in the aged silver-doped hydroxyapatite sol for 5 seconds. The scaffold is then removed from the sol and centrifuged for 10 seconds at 1000 rpm to remove excess sol. The silver-doped hydroxyapatite sol-coated scaffold is immediately baked and dried for 5 hours at 70° C., followed by a heat treatment at 650° C. for 3 hours using the following 3-step heating profile (FIG. 12):
Step 1: heat 3° C./minute until 650° C.
Step 2: keep 650° C. for 3 hours.
Step 3: cool 3° C./minute to room temperature.

### Example 3: Examples of Properties of Hydroxyapatite Scaffolds

The materials discussed below were prepared by the methods of Example 1 and Example 2.

### 3.1 Polymeric Sponge Template

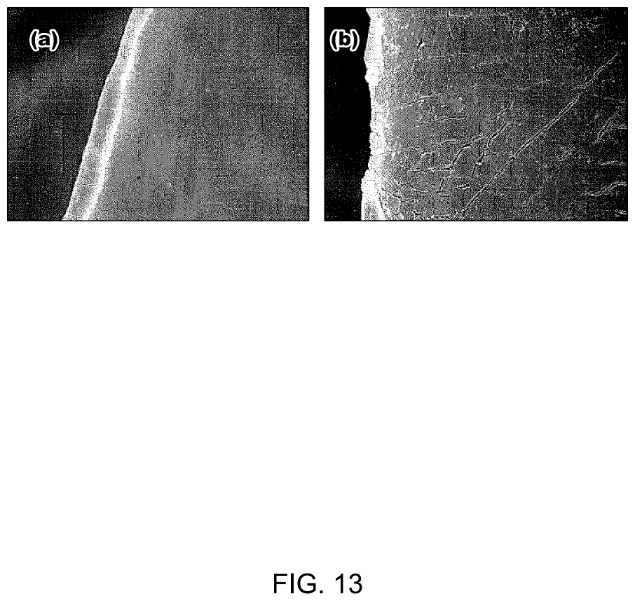

After 20 minutes of treating the polymeric sponge template with 10% sodium hydroxide, the surface of the sponge is changed from smooth to rough and is more hydrophilic. As shown in FIG. 13, the shape of the originally cut sponge remains intact, together with its elastic property.

### 3.2 First Time Calcium Phosphate Coated, Dried and Sintered Scaffold

After immersing the treated polyurethane sponge template in a first time coating slurry followed by drying at 27° C. in a 80% humidity environment for 60 hours, the bi-layered calcium phosphate coated sponge template appears hardened. The coated surface also appears dense and smooth, with only a few cracks observed. Additionally, the scaffold shrinks by 8%.

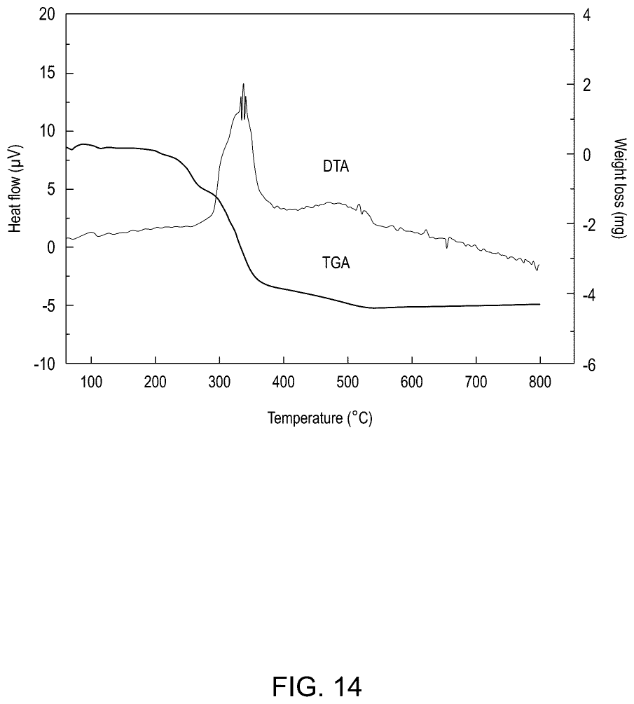

After sintering the coated sponge using an 8-step sintering profile shown in FIG. 9, the resulting scaffolds appear strong, with uniform coating and well interconnected. The sintered scaffold shows 87% porosity as measured using a gas chromatography method. The compressive strength of sintered scaffold is in the range of the compressive strength of the human cancellous bone (2-180 MPa). As shown in FIG. 14, the TG/DTA curve of polyurethane sponge indicates that the polyurethane sponge burn out occurs from 230° C. Violent burn out of the sponge occurs at temperature from 280° C. to 400° C., with the triangular scaffold struts remaining interconnected after the sponge burn out. Length of the triangular secondary micro-channels inside the strut is 40 μm on each side. During this temperature range, the powders in the slurry become semi-molten, thereby allowing viscous flow of the powders and resulting in neck formation between powders.

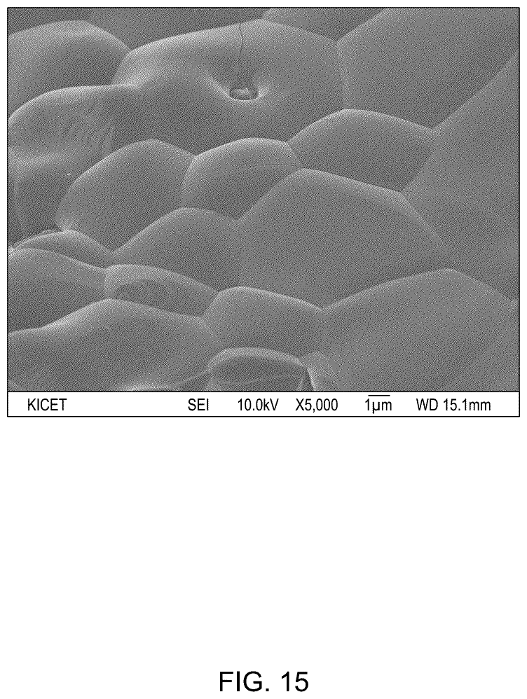

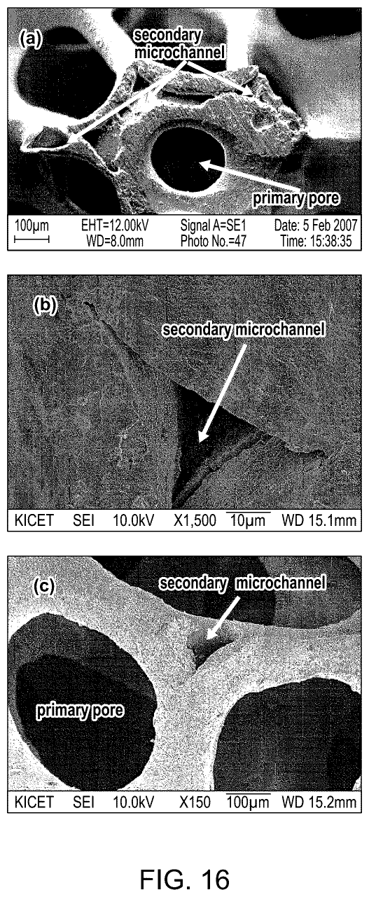

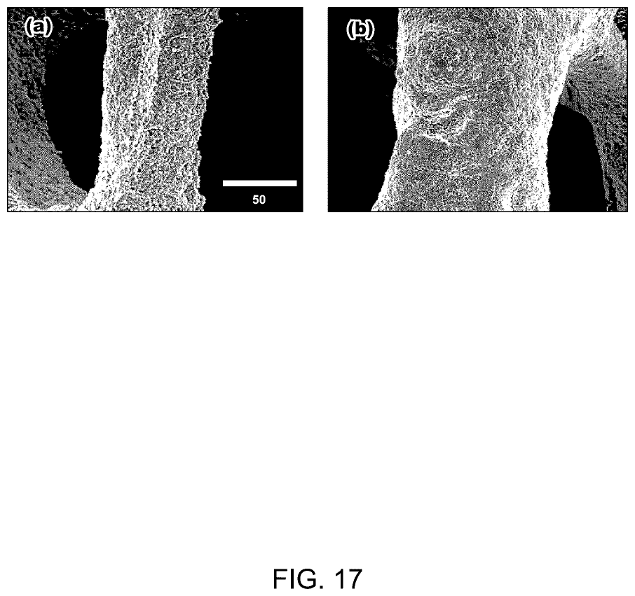

At temperatures between 400° C. and 600° C. and between 600° C. and 1230° C., the calcium phosphate-coated layer interconnects the pores and coarsened the coated surfaces, respectively. Densification of the coated layer occurs when scaffold is sintered at 1230° C. for 3 hours. After sintering, the calcium phosphate scaffolds appears to shrink by 22%. The sintered scaffold surface is dense and smooth, with shown clear grain boundaries as observed using a scanning electron microscopy (FIG. 15). Cross-sectioning of the scaffold shows the presence of triangle shape secondary micro-channels within the triangular struts (FIG. 16). The function of these secondary micro-channels is to allow the transport and diffusion of nutrients and waster when the scaffold is implanted in the human bone. These features allow the regenerated bone tissues to be kept alive and functional over time.

### 3.3 Second Time Calcium Phosphate Coated, Dried and Sintered Scaffold

After immersing the first time coating and sintering in the second time coating slurry, followed by drying at 30° C. in a 70% humidity environment for 24 hours, the coated surface appears dense and smooth, but is slightly thicker than first time coated surface (FIG. 17). Sintering of the scaffold using a 5-step sintering profile (as shown in FIG. 10) occurs after drying, with the scaffold shape and size remaining intact after shrinking. No additional shrinkage occurs during the second time coating process. Additionally, there is no change in the size of the secondary micro-channels. The final grain size of sintered scaffold surface remains the same as the observations made on the first time coating and sintered scaffold.

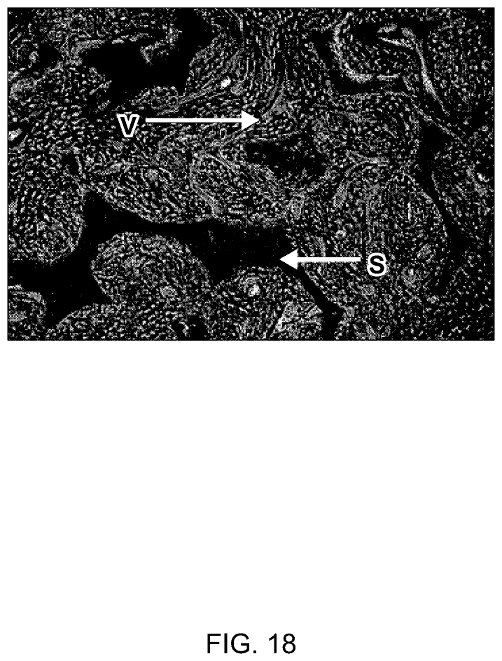

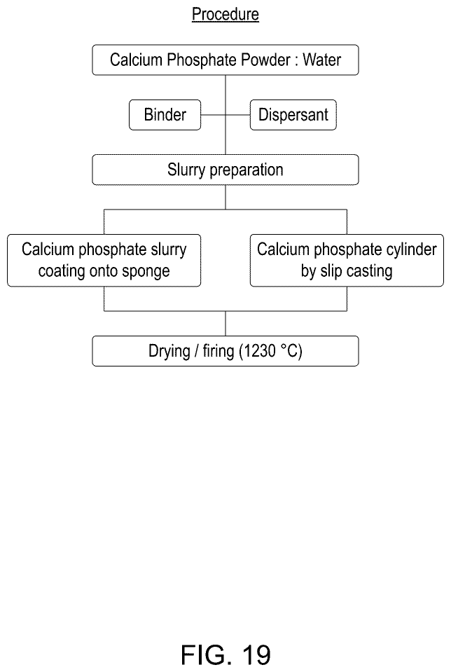

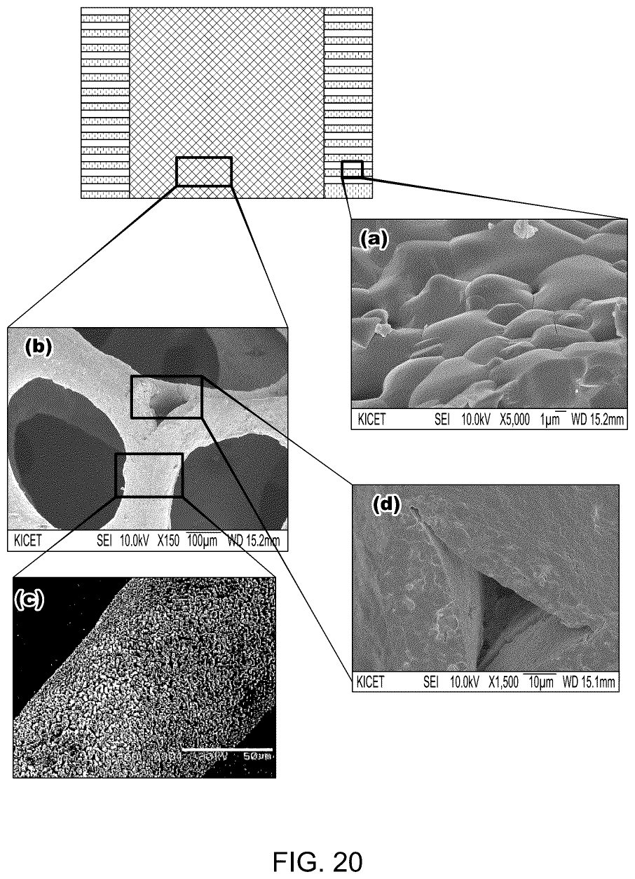

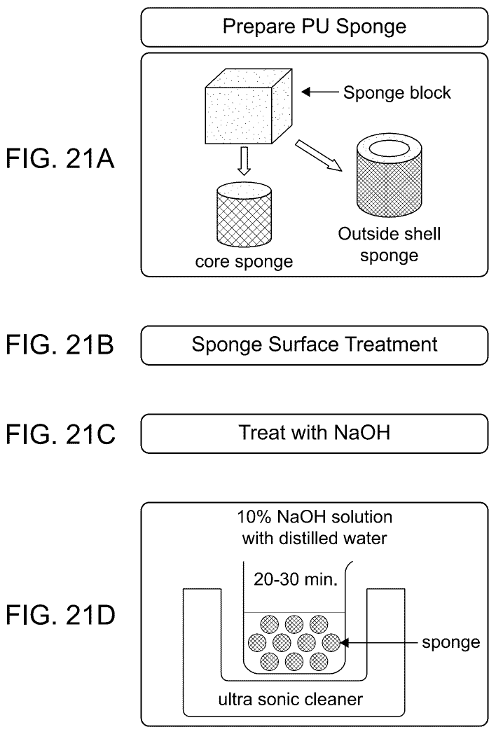

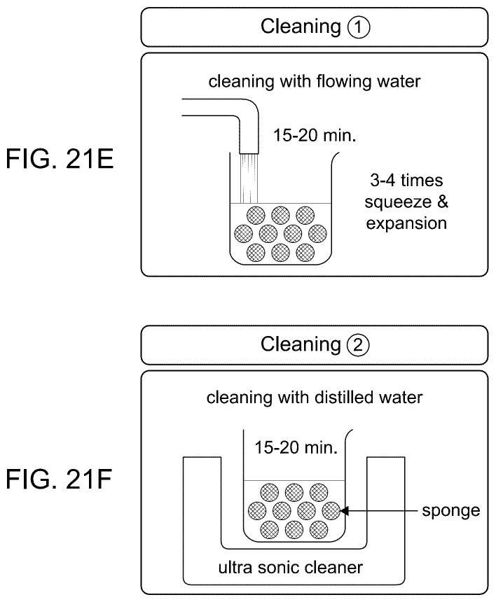

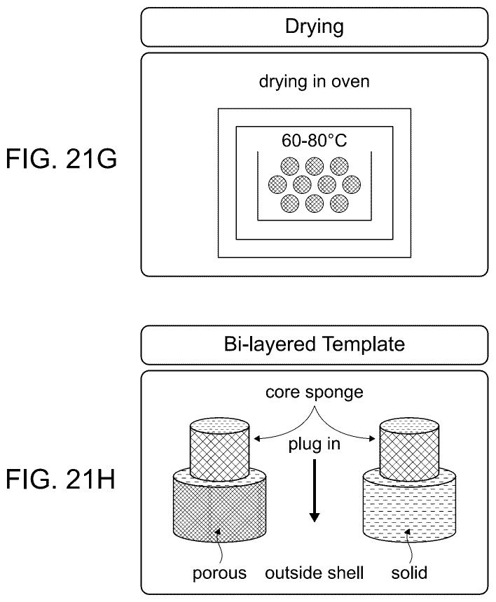

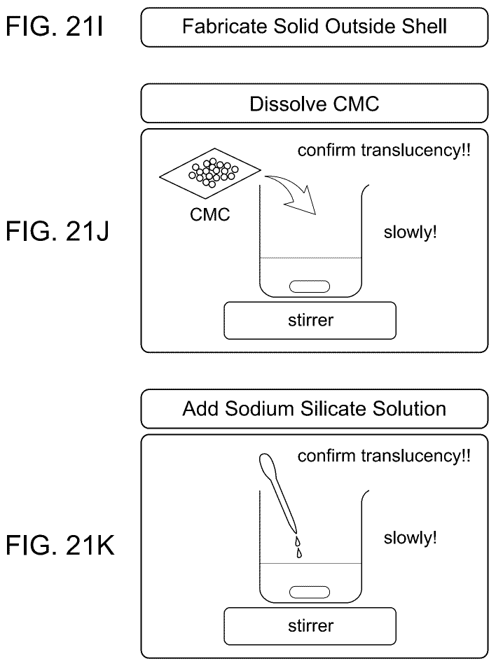

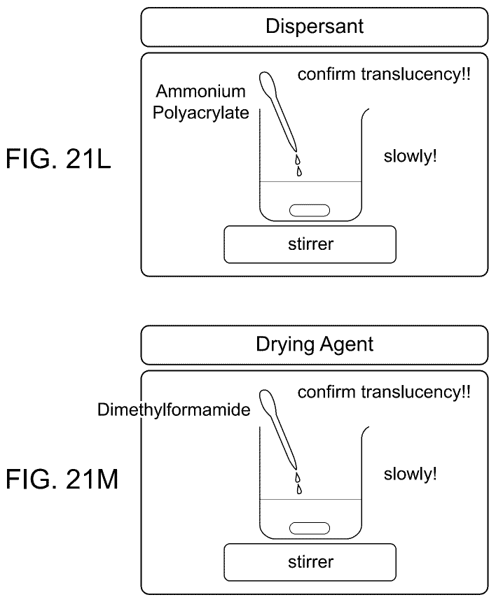

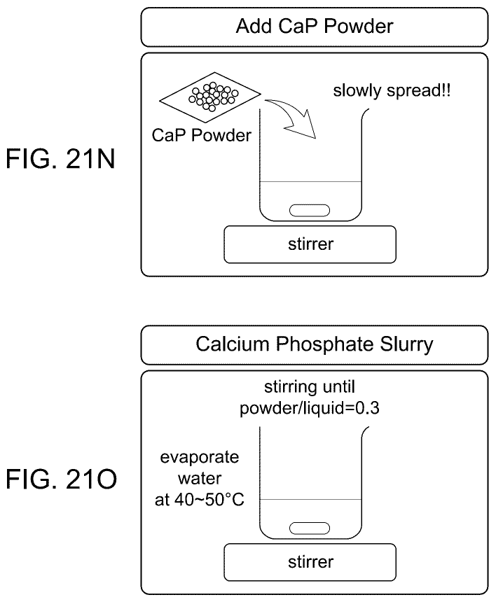

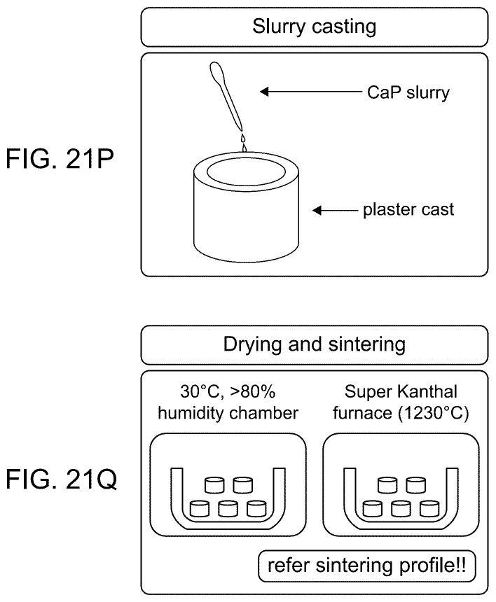

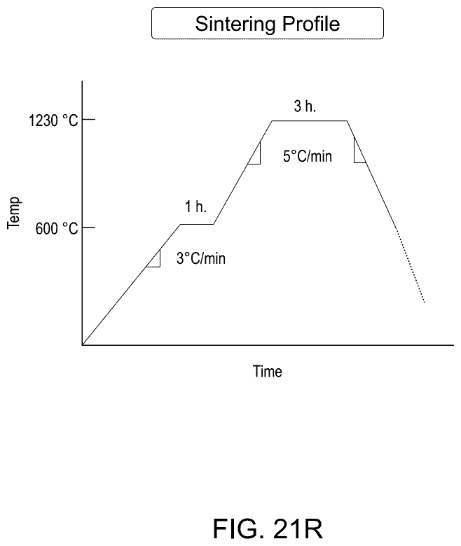

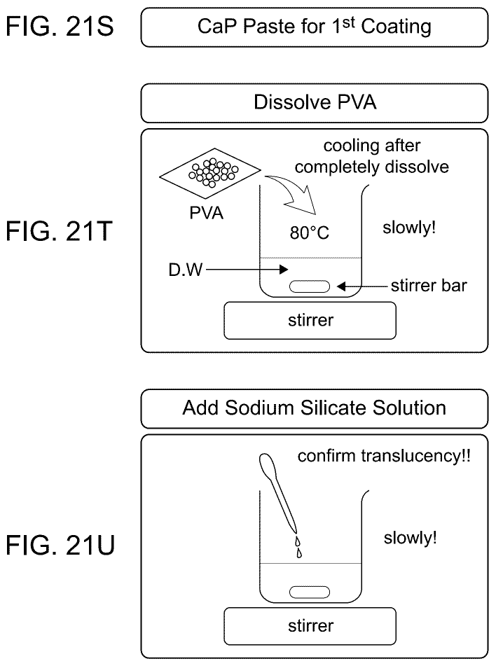

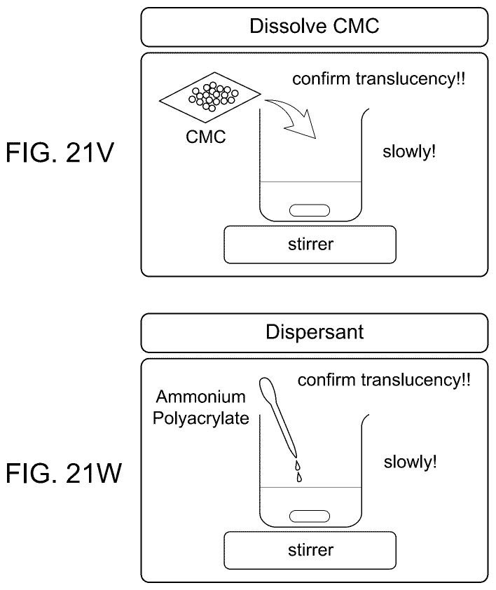

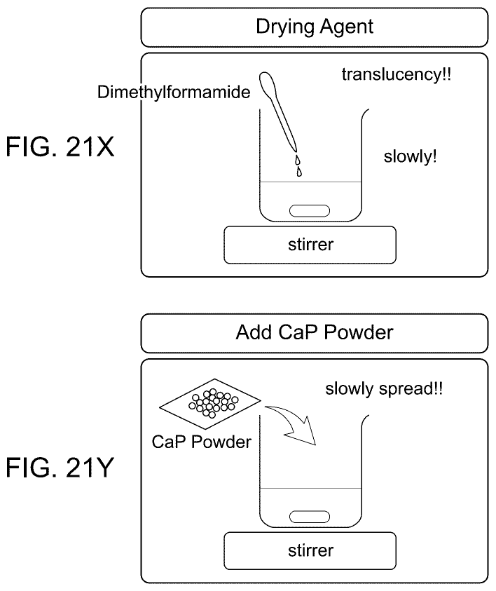

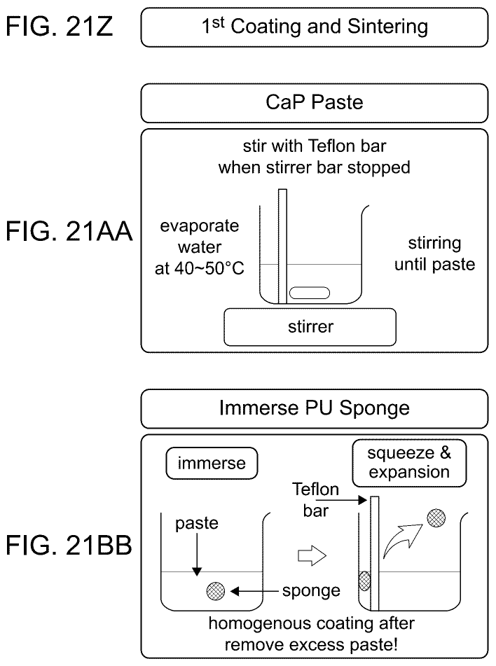

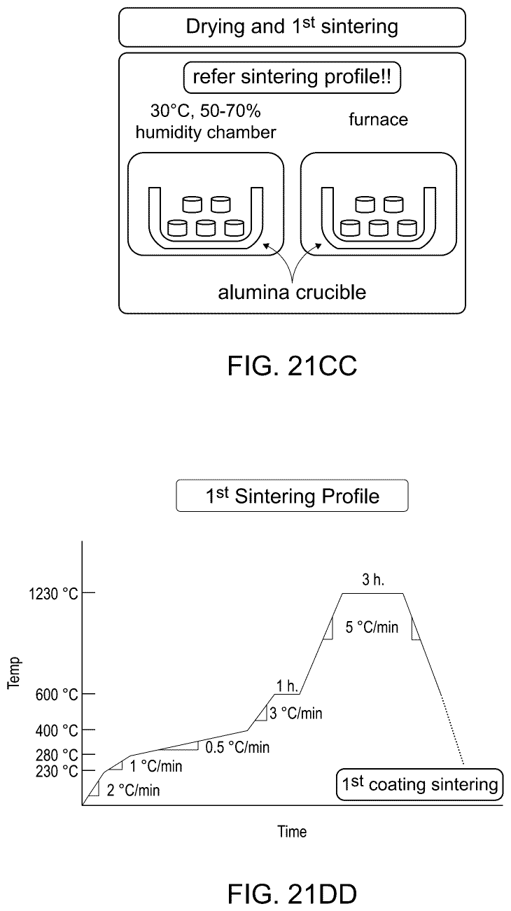

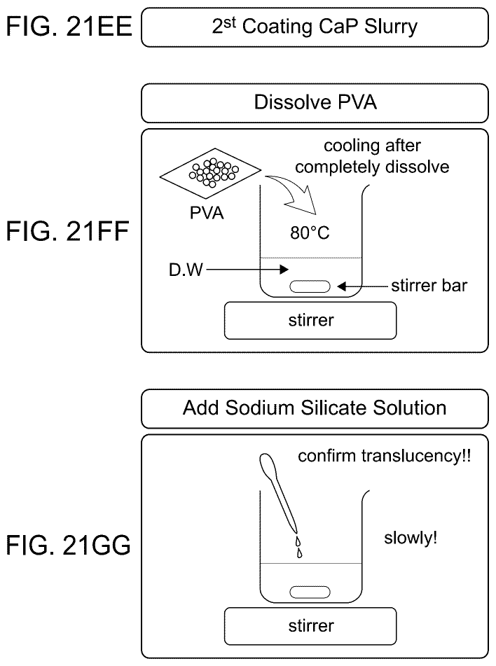

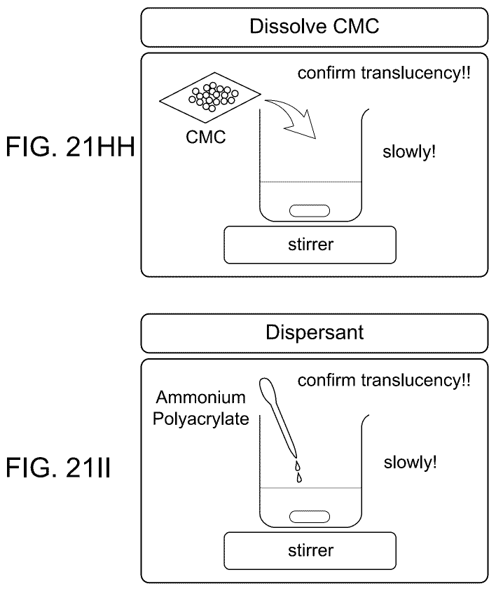

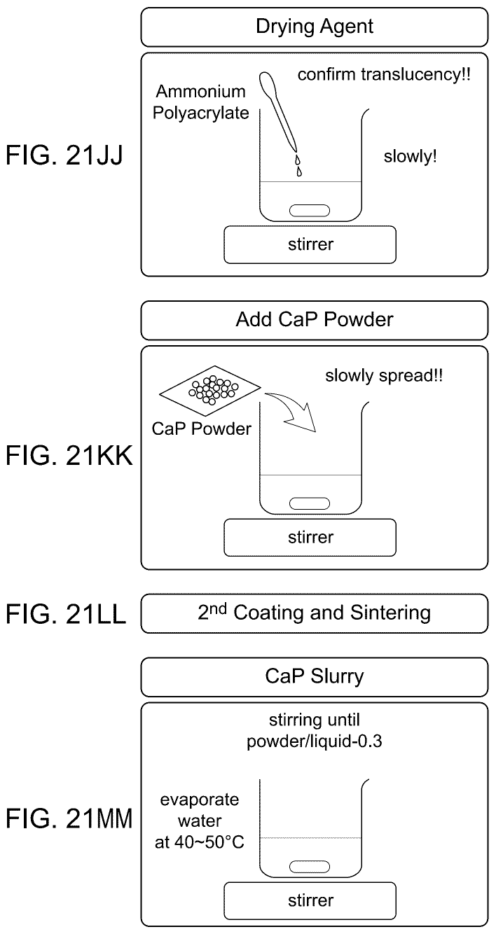

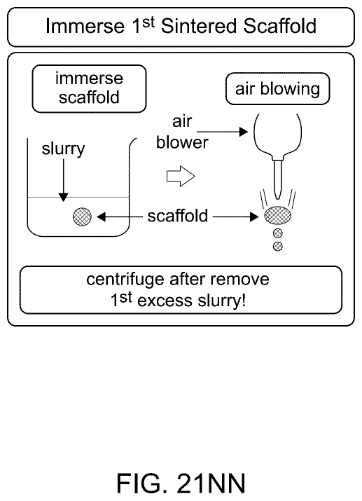

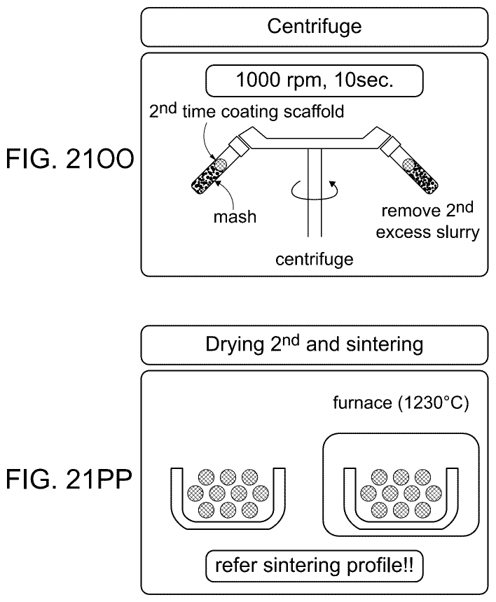

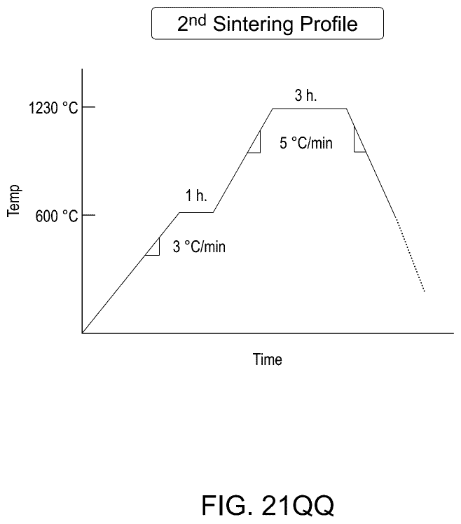

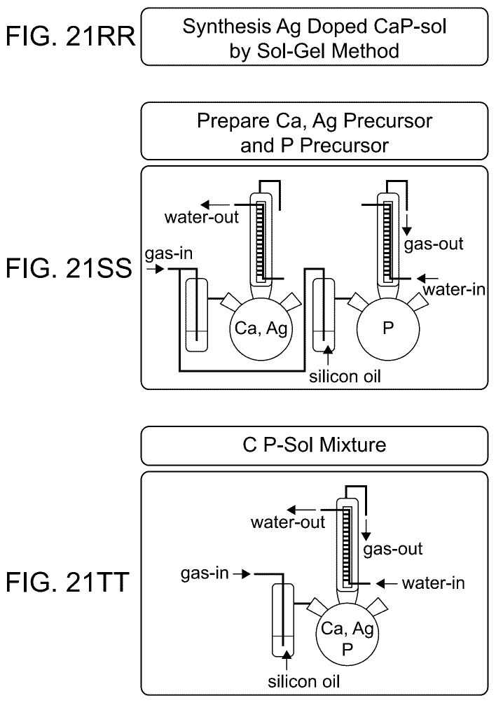

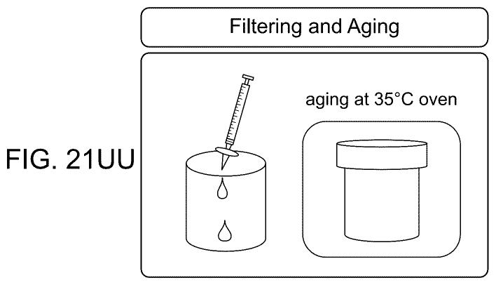

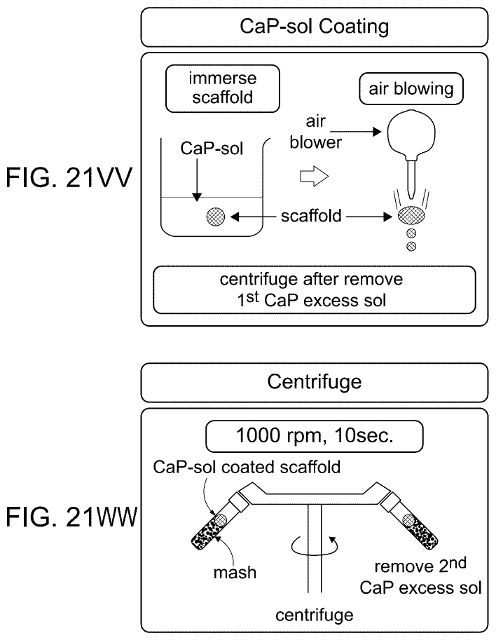

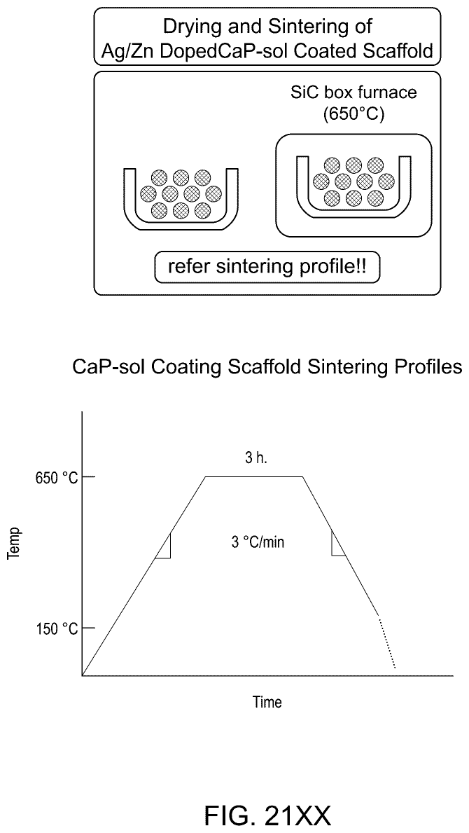

The triangular-shaped strut observed during the first time coating and sintering process becomes rounded after the second time coating and sintering process (FIG. 17). This rounded strut shape is friendlier for encouraging bone or osteoblast cells to attach on the scaffold surface when compared to the triangular-shaped strut. Additionally, in this invention, the complete interconnectivity and uniformity in the pores allow bone/osteoblast cell migration to the center of scaffold. The ability to allow cells to migrate throughout the entire scaffold also means that communications between bone/osteoblast cells in the scaffolds are not hindered. In addition to the complete interconnectivity and uniformity in the pores, continuous secondary micro-channels within the struts also allow the transport of blood, nutrients, and wastes between the implanted scaffold and natural bone as well as within the scaffolds. These functional structures (interconnectivity and uniform pores as well as secondary micro-channels) also allow the bridging of the scaffolds to the natural bone by the bone/osteoblast cells and vascular ingrowth (FIG. 18).

### 3.4 Antibacterial Silver-Doped Hydroxyapatite Sol Coating, Drying and Sintering

Antibacterial silver-doped hydroxyapatite sol coating is performed after the second time coating and sintering process. No change in shape, structure, and mechanical strength occurs after the coating process, drying at 70° C. for 5 hours in still air environment, and heat-treated at 650° C. for 3 hours using a 3-step heat treatment profile (as shown in FIG. 12). When the silver-doped hydroxyapatite sol is coated on a 2-dimensional metallic implant surface, low or minimal bacteria adhesion is observed when compared to the non-coated or non-silver-doped hydroxyapatite coatings: thus, the silver-doped hydroxyapatite sol coating on the 3 dimensional scaffolds of the present invention will similarly provide a strong antibacterial property. Zinc-doped hydroxyapatite sol coating on scaffolds will have the same antibacterial property.

### Example 4: Fabrication of a Three-Leveled Scaffold Structure

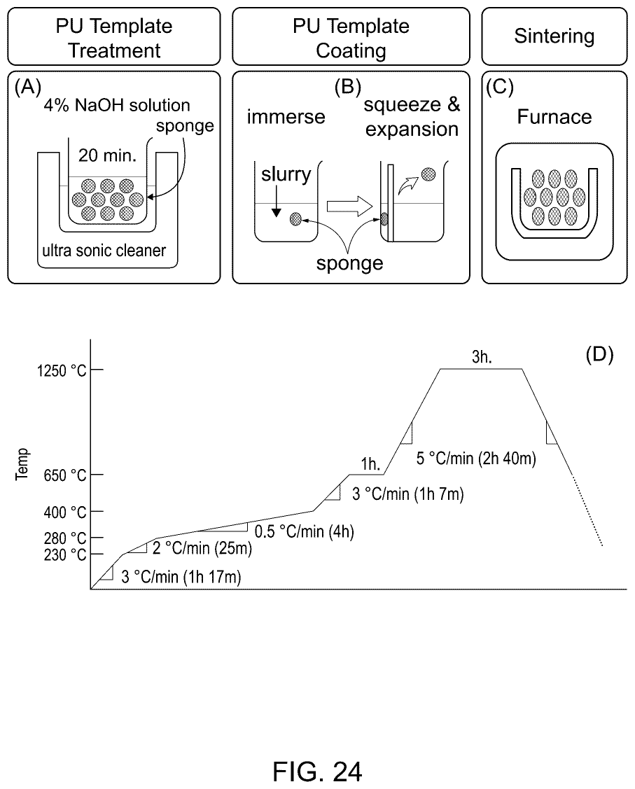

In order to mimic the fluid/gas-conducting channels found in natural bones, micro-channels were engineered within the trabecular septa of our scaffolds. A polyurethane (PU) template (60 pores per inch (ppi): E.N. Murray Co., Denver, CO) was coated with nano-sized hydroxyapatite (HA) powders (HA: OssGen Co., Gyeongsan, Korea) in a distilled water-based slurry. Because PU templates have a pre-defined porosity, the size of the scaffold primary macro-pores, dimensions, and geometry could be adjusted by selecting PU templates of different pores per inch. To complete a homogeneous and continuous capillary structure, the fabrica-tion steps were precisely controlled throughout the entire procedure. The PU templates were pre-treated with 4% NaOH solution for 20 min in an ultra-sonicator to modify the surface property and then dried at 40 1 C in an oven. The 25 ml of coating solution mixed with 3 wt % polyvinyl alcohol (89,000-98,000 Mw, Sigma-Aldrich, USA), 1 wt % sodium carboxymethyl cellulose (ultra low viscosity, Sigma-Aldrich, USA), 3 wt % ammonium polyacrylate dispersant (Darvan 821, R.T. Vanderbilt, USA), and 2 wt % Glycerin (Sigma-Aldrich, USA) was prepared. The amount of the reagent was measured versus powder content, respectively. 8 g of the HA nano-sized powder was slowly added to the coating solution while stirring and heating to condense until the powder/solution ratio reach to 1.77-1.80. The treated PU template was immersed into the coating slurry and squeezed a couple times until the slurry coated the PU template homogenously. The amount of slurry to coat one PU template was eight-fold of the template by weight. The excess slurry was removed by using low air pressure. This also ensured the integrity of the macro-pores in the scaffold. The coated template was dried overnight under cooling conditions (20-25 1 C) with gentle air circulation. Finally, the completely dried specimens were sintered according to 8-step heat treatment procedure in a high temperature furnace at 1250 1 C for 3 h (see FIG. 24). The PU template was incinerated during the sintering procedure, thus leaving behind a void we define as micro-channels. On the other hand, the HA slurry that had coated the PU template solidified to become dense trabecular septa.

### Example 5-Fabrication of Micro-Channeled (MC) Scaffold

Fully interconnected porous MC-scaffolds were fabricated using a polyurethane (PU) template (60 pores per inch: E.N. Murray Co., Denver, CO, USA) coating method. In order to fabricate a scaffold with fluid/gas-communicating channels that are observed in natural bones, a PU template was used to make channels within the trabecular septa. The PU template was coated with nano-sized hydroxyapatite (HA) powders (OssGen Co., Gyeongsan, Korea) in a distilled water-based slurry. To accomplish a homogeneous and continuous engineered micro-channel structure, the fabrication steps were precisely controlled throughout the entire procedures. Briefly, (1) the PU templates were pre-treated with 4% NaOH then dried in an oven at 40° C. for next use. (2) A 25 ml of viscous solution mixed with 3% polyvinyl alcohol (89,000-98,000 Mw, Sigma-Aldrich, USA), 1% sodium carboxymethyl cellulose (ultra low viscosity, Sigma-Aldrich, USA), 3% ammonium polyacrylate dis-persant (Darvan 821, R.T. Vanderbilt, USA), and 2% Glycerin (Sigma-Aldrich, USA) was prepared. (3) HA powder was slowly added to the viscous solution while stirring and heating to condense until the powder/solution ratio reach to 1.77-1.80. (4) The pretreated PU template was immersed into slurry and squeezed to complete homogeneous coating. (5) Slightly blown air pressure was used to remove excess slurry that covered the macro-pores. (6) The coated template was dried overnight under cooling conditions (20-25° C.) with gentle air circulation. (7) Finally, the completely dried specimens were sintered in a high temperature furnace at 1,250° C. for 3 h.

### Example 6—PCT/Template Preparation

### 1. Polyurethane (PU) Sponge Preparation as Template

(1) Use PU sponges to produce hydroxyapatite templates containing interconnecting pores. Use each sponge to provide the primary trabeculae for the formation of the template struts as well as the formation of micro-channels within the trabeculae.
(2) Cut and trim 80 ppi (pores per inch) sponges into 2 bridge-shapes with dimensions of 3.5 cm in height×5 cm in length×1.5 cm in width. Note: The dimensions and shapes can be chosen according to the desired primary pore size: 100 ppi, 80 ppi, and 60 ppi.
(3) Make a 100 ml of 4% (w/v) NaOH solution using a 150 ml beaker; then immerse and squeeze until the prepared sponges are completely soaked.
(4) After soaking, place the beaker with the sponges in the ultrasonicator (42 kHz).
(5) Ultrasonically pre-treat the PU sponges for 15-20 min without heat to modify the surface properties.
(6) Rinse with distilled water for 5-10 min. While rinsing, squeeze the sponges and allow them to expand 5 to 7 times in order to remove the residual NaOH inside the sponges.
(7) Squeeze the sponges with paper towels in order to remove excess water; then dry them in an oven at 60-80° C.

### 2. Hydroxyapatite (HA) Slurry Preparation for Coating

(1) Before making the HA slurry, measure the weight of a beaker with a magnetic stir bar. This measurement will be used to calculate the powder/liquid ratio.
(2) Measure 10 g of the nano-sized HA powder.
(3) Add 20 ml of distilled water into the 50 ml beaker. Heat for 120-140° C. and stir using a hot plate magnetic stirrer.
(4) Add 0.3 g (3% w/w) of polyvinyl alcohol (PVA) (89,000-98,000 MW) per powder into distilled water while stirring at 300-400 rpm.
(5) Stir until the PVA has completely dissolved. The solution should be clear after complete dissolution of the PVA.
(6) Turn off the heat and add 0.1 g (1% w/w) of sodium carboxymethyl cellulose (CMC) (ultra-low viscosity) for powder while stirring at 400-500 rpm. The solution should be clear after complete dissolution of the PVA.
(7) Stir until the CMC has completely dissolved and cool down to RT.
(8) Add 0.3 g (3% w/w) of ammonium polyacrylate dispersant per powder while stirring at 300-400 rpm. Stir until completely dissolved.
(9) Add 0.2 g (2% w/w) of glycerin per powder while stirring at 300-400 rpm. Stir until completely dissolved.
(10) Slowly disperse the HA powder into the solution while stirring at 600-900 rpm and keep stirring for 5 min.
(11) Sonicate for 5 min using an ultrasonicator to ensure dispersion of any agglomeration of the HA powder.
(12) Add an extra 5 ml of distilled water into the mixture while stirring at 600-900 rpm and heat at 90-100° C.
(13) Keep stirring the mixture using a magnetic stirrer at 600-800 rpm at 90-100° C. in order to evaporate the water content.
(14) Measure the whole weight including the beaker and mixture from time to time until a powder/liquid ratio of 1.75-1.8 is obtained.
(15) Formatting the powder/liquid ratio, divide the weight of the powder by the total weight of the mixture (2.14), including the powder, reagents, and water, minus the weight of the beaker and stirrer (2.1), and minus the HA powder (2.2).
   Note: For example: If A (entire mixture including powder, reagents, and water) is 49.05 g, B (beaker with stirrer) is 33.5 g, and then C (HA powder) is 10 g.

$$C / ( A - B - C ) = 10 / ( 49.05 - 33.5 - 10 ) = 1.8$$

(16) Allow the slurry to cool down to RT before using for coating.
   3. HA coating, Drying, and Sintering
(1) Coat the prepared PU sponges with the HA coating slurry using a stainless spatula until the slurry is homogenously distributed throughout the PU sponge onto a glass plate.
   Note: After removing the excess slurry, some of pores may still be clogged with slurry because of the high slurry viscosity.
(2) In order to ensure interconnectivity, uniformity, and open pores, slightly blow the HA coated templates using an air compressor. This process ensures that the templates are homogeneously coated both on the inside and outer surfaces of the PU sponge.
   Note: If a homogeneous coating is not achieved, the HA coated templates will collapse during the sintering process and may also crack while handling due to low mechanical strength. Additionally, the homogeneous coating is critical in creating micro-channels within the trabeculae.
(3) Dry the HA coated templates for a minimum of 5 hr under cooling conditions (20-25° C.) with gentle air circulation. However, extend the drying time based on the size of template.
   Note: After drying, the HA coated templates will typically shrink approximately 8% to 10% in each dimension.
(4) After the drying process, place the HA coated templates on an alumina crucible. Then, place them in a high temperature furnace and use the following 8 step sintering profile.
   (i) Heat 2° C./min until 230° C.
   (ii) Heat 1° C./min until 280° C.
   (iii) Heat 0.5° C./min until 400° C.
   (iv) Heat 3° C./min until 600° C. Keep at 600° C. for 1 hr.
   (v) Heat 5° C./min until 1230° C. Keep 1,230° C. for 3 hr.
   (vi) Cool 5° C./min to RT.
      Note: Sintering will further shrink the HA coated sponge templates by approximately 22%-25% in each dimension.
      4. Ingress and Inhabitancy of Cells into Template
(1) Culture pre-osteoblastic MC3T3 cells in a non-osteogenic media consisting of α-MEM, supplemented with 10% Fetal Bovine Serum (FBS) and 1% antibiotics (streptomycin and penicillin) at 37° C. in a humidified atmosphere containing 5% CO₂.
(2) Add 10 ml of a cell suspension at 2×10⁶ cell density into a single well within a 6-well plate.
(3) Place the 3 cm×4 cm×1 cm bridge-shaped template vertically into the 6-well plate. Place one leg of the template into the plate containing the cell suspension, and the other leg into an adjacent empty well.
(4) Allow the template to absorb the cell suspension for 10 min.
(5) Add 5 ml of media thereafter to the well that was originally filled with the cell suspension.
(6) Replenish the medium in both wells every 2 or 3 days until 7 days have elapsed.
(7) Determine the efficacy of cell mobility by hematoxylin and eosin staining.
   (i) Fix the cells and scaffold by immersing in 100% EtOH for 20-30 min.
   (ii) Stain with Hematoxylin for 1-2 min.
   (iii) Rinse with distilled water for 1-2 min for twice.
   (iv) Dehydrate by immersion in 70%, then 95%, then 100% EtOH for 1-2 min each.
   (v) Stain with Eosin for 20-30 sec.
   (vi) Rinse with distilled water for 12 min for twice.
   (vii) Dehydrate by immersion in 70%, 80%, 90%, and 100% EtOH for 1-2 min each.
   (viii) Embed the scaffold in an acrylic resin for sectioning and imaging.
(8) Determine the cell viability with the 3-[4,5-Dimethylthiazol-2-yl]-2,5-diphenyl tetrazolium bromide (MTT) cell viability assay and Live/Dead assay (Live/dead cell staining kit MPTP) at time points of day 3 and 7.
   Note: The scheme of bone-like template fabrication protocols are represented in the “Representative Results” section below.
(9) Representative Results.

The overall structure of the BMT exhibits a unique three-dimensional template with trabecular bone-like internal structures. The BMT contains macro-pores, micro-channels, and nano-pores. Clear configurations of fully interconnected macro-pores (average size of 320 μm), micro-channels (average diameter of 50 μm), and nano-pores (average size of 100 nm) were verified with a scanning electron microscope (EVO-40; ZEISS) as well as through micro-tomography.

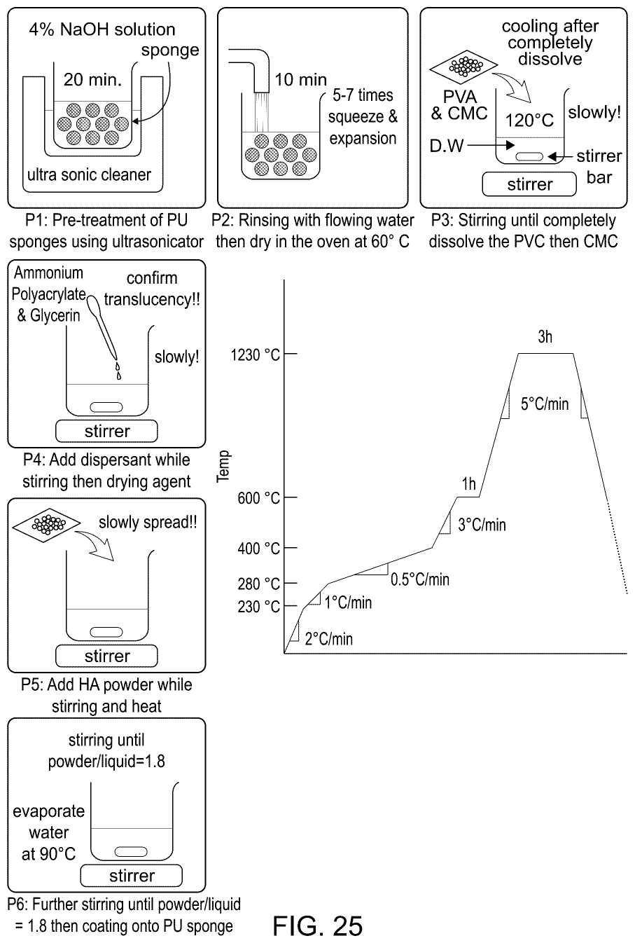

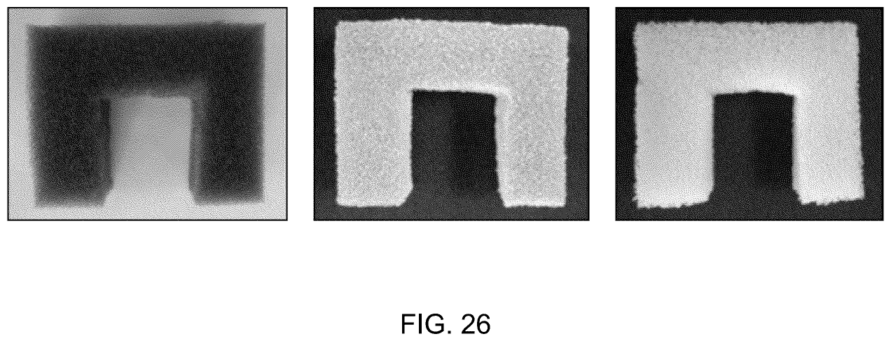

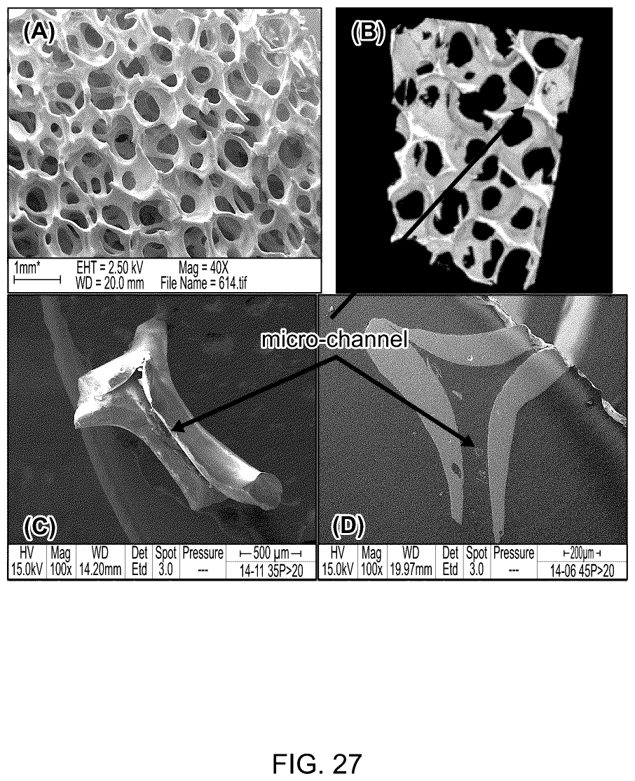

FIG. 25 shows stepwise detailed protocols in creating a BMT. Through precise control of the protocols from the preparation of the PU sponges to the sintering process (Steps P1-P7 in FIG. 25), the following features can be achieved: a highly dense and smooth surface after HA coating and drying; a precisely shaped and sized 3-D template; a fully interconnected porous trabecular network similar to that of trabecular bone; and micro-channels within each trabecula that mimic intra-osseous channels such as Haversian canals and Volkmann's canals (see FIGS. 26 and 27).

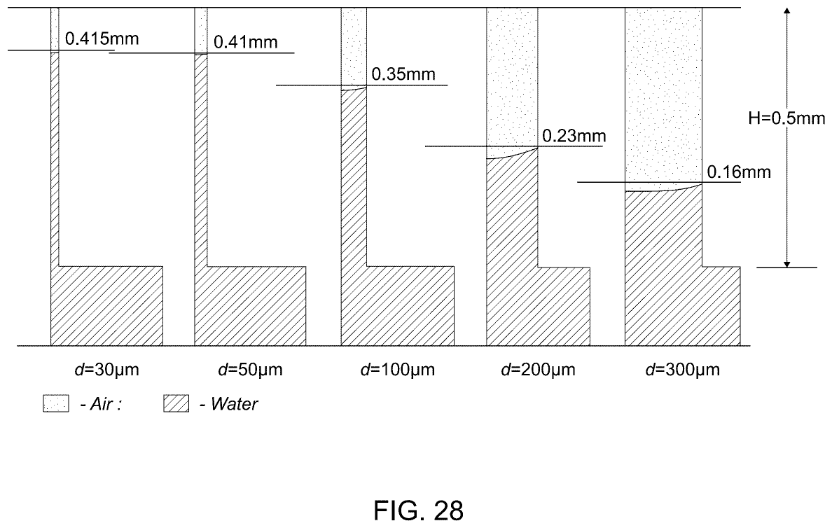

Furthermore, relatively high mechanical strength (˜3.8 MPa) similar to that of human trabecular bone was measured by a compressive strength test. Highly similar histomorphometric parameters with those of human lumbar vertebrae trabecular bone were confirmed by micro-CT analysis. Different magnitudes of capillary action were demonstrated through different capillary diameters in FIG. 28 using computational simulation.

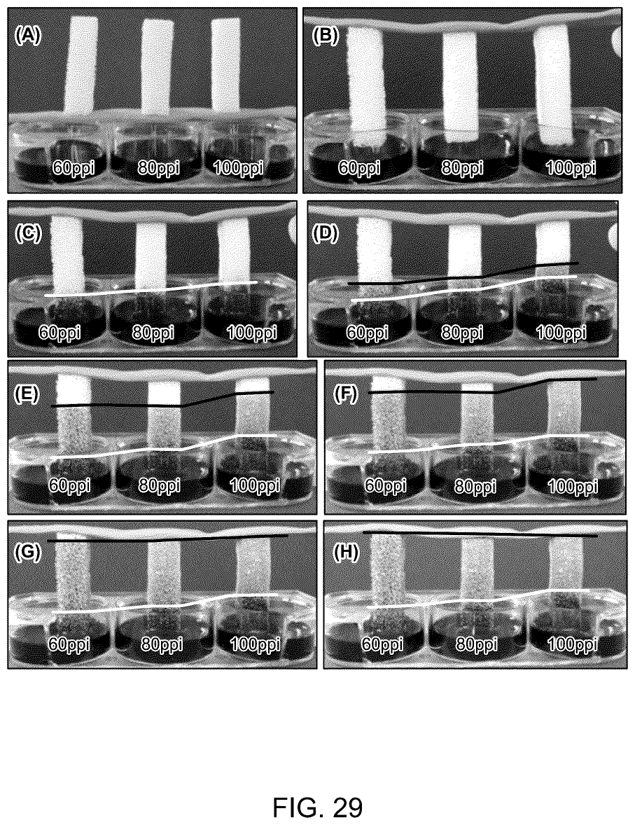

Through these simulations, we projected that the BMT would exhibit varying absorption rates within the primary-pores (300-400 μm) and micro-channels (25-70 μm) based on the diameters. Smaller capillaries exhibited stronger absorption capacities. This assumption was verified in this experiment as shown in FIG. 29.

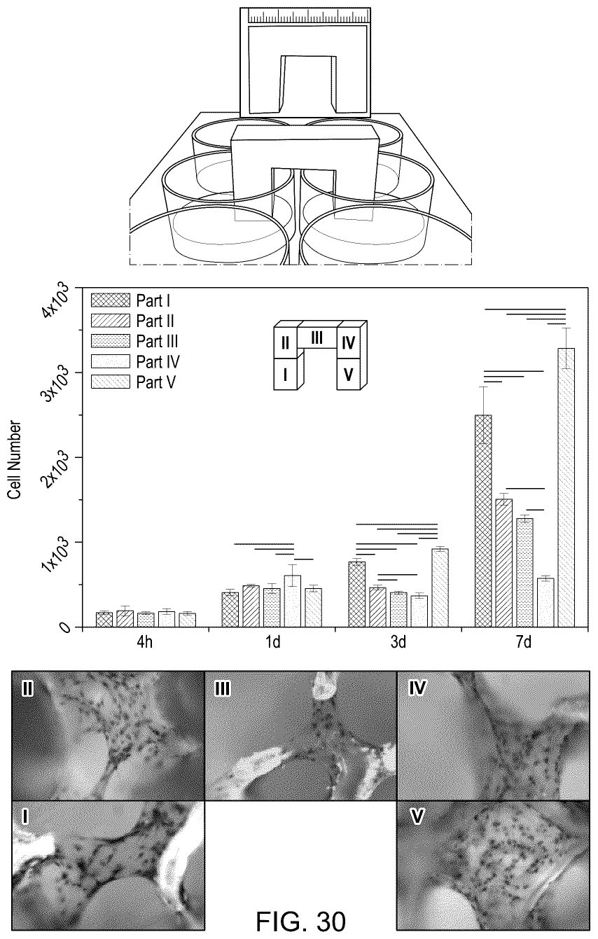

The BMT exhibited highly effective fluid absorption and retention through the capillary action of the micro-channel structures; stevenel's blue stain was used as the fluid medium to easily track the flow FIG. 29. Based on computational simulation, the BMT with these configurations were seen to absorb and retain cell suspensions up to 8.5 cm in total distance within 10 sec. Due to a strong capillary action induced by the internal structures, the stained medium reached the opposite end of a 3 cm (height)×4 cm (length)×1 cm (width) bridge-shaped template within 1 min and 40 sec. Furthermore, active cell mobilization and incorporation into the BMT was observed FIG. 30. Subsequently, the homogenous cell mobilization and attachment resulted in enhanced proliferation and matrix formation in an evenly distributed formation. Moreover, long-distance (˜ 10 cm) migration of cells through the BMT was validated immediately after the BMT was saturated with the cell suspension.

Seeded cells survived in the template segment that was exposed to the air and not immersed in the culture medium. In this experiment, the culture medium was provided to the cells by exclusively in the wells touching only the legs of the scaffold. The capillary action exhibited by the micro-channels then allowed for fresh medium to reach the top, bridge portion of the scaffold. After 3 days of culture, the template became occupied with rapidly proliferating cells. After 7 days of culture, each trabecula was wrapped by extra cellular matrices and embedded with cells.

### Bioreactor System

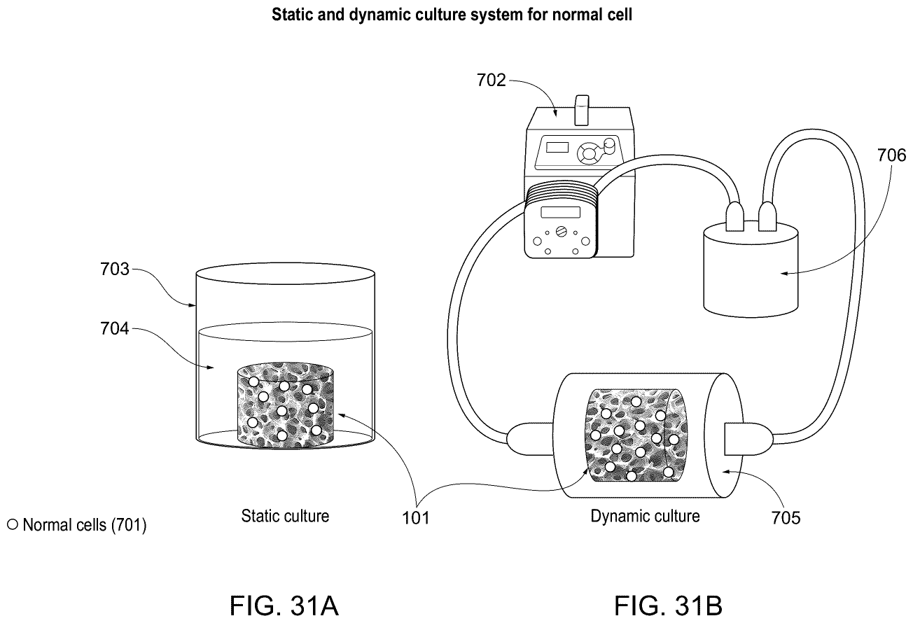

Referring now to FIG. 31, a bioreactor system suitable for primary culture of normal cells is depicted according to embodiments of the present disclosure. FIG. 31A depicts a static culture system for normal cells. A PCT 101 is placed at the bottom of a 12-well culture plate 703. The PCT 101 is seeded with normal cells 701 in a conventional manner. A culture medium 704 is added into the culture plate 703 in which the PCT 101 is placed. Afterwards, static culture is executed for up to, but not limited to, about five days. In an exemplary embodiment, the normal cells 701 include osteoblast precursors with a cell population of about 2.5×10⁵. However, the present disclosure is suitable for culture of various cells and cell populations including precursors such as mesenchymal stem cells, chondrocytes, osteoblasts, osteoclasts, fibroblasts, muscle cells, bone marrow cells or a combination of two or more of the foregoing cells, and up to, but not limited to, cell populations of about 3×10⁶.

Referring now to FIG. 31B, it depicts a dynamic culture system for normal cells according to embodiments of the present disclosure. After the performance of the static culture illustrated in FIG. 31A, the PCT 101 is inserted to a culture column 705 to apply a dynamic culture system using a peristaltic perfusion pump 702. The PCT 101 is placed in the culture column 705 that is connected with a culture medium vessel 706. The dynamic culture is continued for up to, but not limited to, about five days. In an exemplary embodiment, a flow rate of the medium through the culture column 705 is about 5 ml/min. However, the flow rate may vary in accordance with maturation of 3D culture model up to or over about 30 ml/min. All fluid flow durations, directions, and times are variable, depending on cells colonization and extra cellular matrix formation in the PCT 101. In an exemplary embodiment, on the first day, forward direction flow may be performed; on the second day, backward direction flow may be performed; on the third day, forward direction flow may be performed; etc.

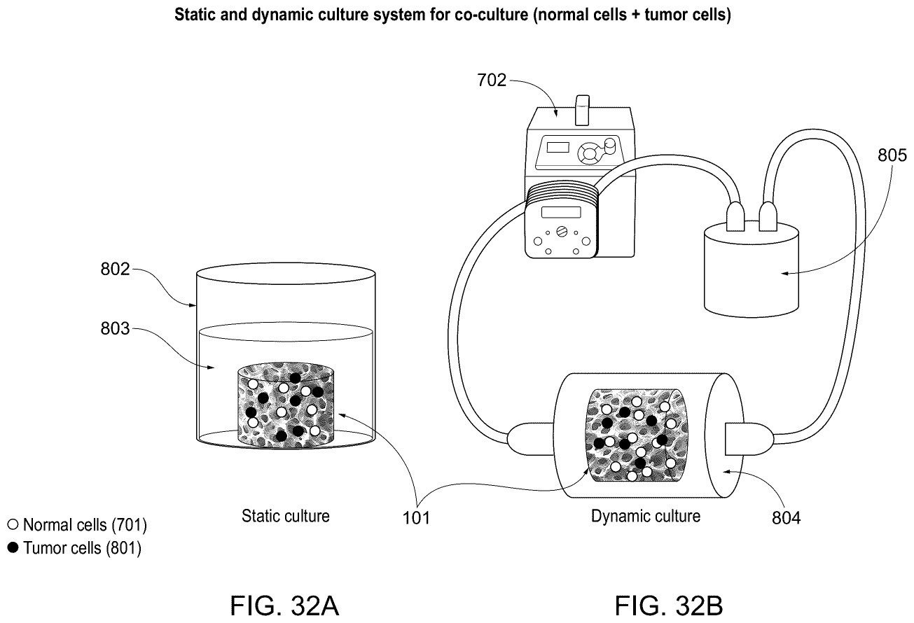

Referring now to FIG. 32, a bioreactor system suitable for co-culture of normal cells and tumor cells is depicted according to embodiments of the present disclosure. FIG. 32A depicts a static culture system for co-culture. A PCT 101 pre-cultured with normal cells 701 with a cell population of about 2.5×10⁵ is placed at the bottom of a 12-well culture plate 802. The PCT 101 is also seeded with tumor cells 801. A culture medium 803 is then added into the culture plate 802 in which the PCT 101 is placed. Afterwards, a dynamic culture is employed for up to, but not limited to, about five days. In an exemplary embodiment, the tumor cells 801 include osteosarcoma with a cell population of about 1×10⁵. However, the present disclosure is suitable for culture of various cells including but not limited to chondrosarcoma, Ewing's sarcoma, fibrosarcoma, and/or breast cancer cells and up to, but not limited to, cell populations of about 2×10⁶.

With reference to FIG. 32B, it depicts a dynamic culture system for co-culture according to embodiments of the present disclosure. After the performance of the static co-culture illustrated in FIG. 32A, the PCT 101 is moved to a culture column 804 to apply a dynamic culture system using a peristaltic perfusion pump 702. The PCT 101 is placed in the culture column 804, which is connected with a culture medium vessel 805. The dynamic culture is continued up to, but not limited to, about five days. In an exemplary embodiment, a flow rate of the medium into the culture column 804 is about 5 ml/min. However, the flow rate may vary in accordance with maturation of 3D culture model up to or over about 30 ml/min. Fluid flow durations, directions, and times are variable, depending on cells colonization, extra cellular matrix formation, and agglomeration of normal cells and tumor cells in the PCT 101. In an exemplary embodiment, on the first day, forward direction flow may be performed; on the second day, backward direction flow may be performed; on the third day, forward direction flow may be performed; etc.

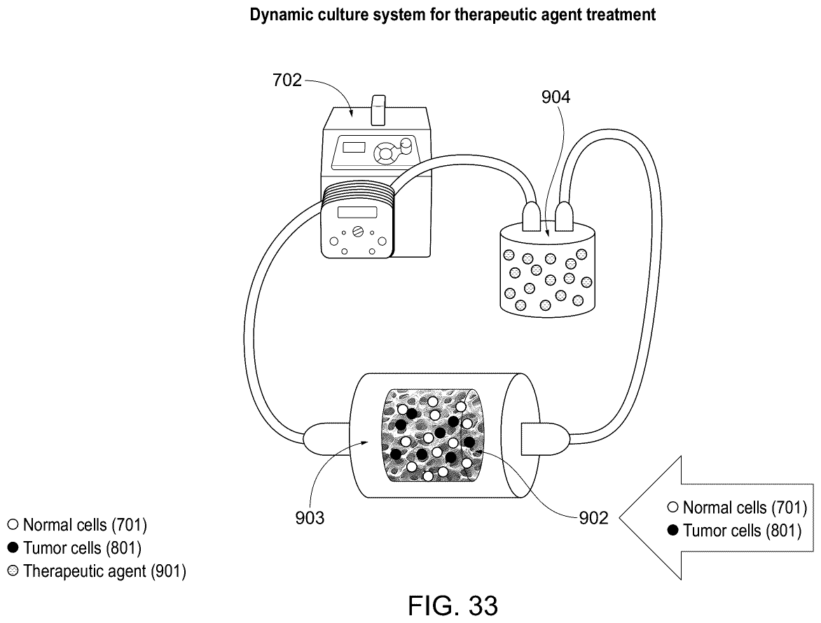

FIG. 33 depicts a bioreactor system suitable for performing a therapeutic agent treatment test according to embodiments of the present disclosure. Reference numeral 902 represents a co-cultured 3D cancer model platform 902 including a PCT 101 (such as the one resulting from the co-culturing discussed above in connection with FIGS. 32A and 32B). The 3D cancer model platform 902 is inserted into a culture column 903 for dynamic therapeutic agent treatment. The culture column 903 is connected with a medium vessel 904 that is filled with a medium in which a therapeutic agent 901 is dissolved. Afterwards, a dynamic therapeutic agent treatment is employed for up to, but not limited to, about seven days. In an exemplary embodiment, the therapeutic agent 901 includes Doxorubicin with a concentration of about 0.5 mg/ml. However, the present disclosure is suitable for cancer treatment with various therapeutic agents including but not limited to Cisplatin, Methotrexate, Ifosfamide, Actinomycin D, Bleomycin, Vincristine, Cyclophosphamide, Paclitaxel, Carboplatin, or a combination of two more of the foregoing agents. In an exemplary embodiment, a flow rate of the therapeutic agent 901 medium into the culture column 903 is about 5 ml/min for about five minutes per day. However, the flow rate and perfusion time may vary in accordance with the efficacy of therapeutic agent treatment up to or over about 30 ml/min for about one hour per day. A fluid flow durations and times are variable, depending on cells decolonization and destruction status of cancel agglomeration in the PCT 902.

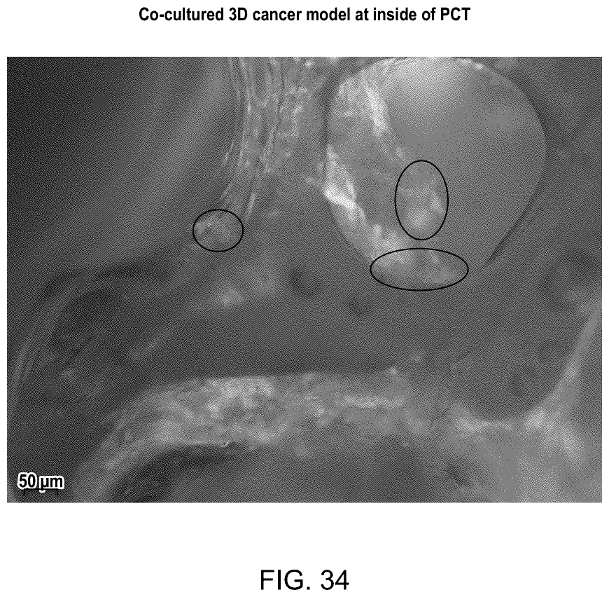

In an exemplary embodiment, FIG. 34 represents a co-cultured 3D cancer model after three days of static culture and three days of dynamic culture using a PCT seeded with osteoblast precursors (MC3T3) and osteosarcoma (143B). MC3T3 osteoblasts were seeded onto a PCT at a concentration of 2.5×10⁵ cells and cultured for three days under normal conditions after static culturing in a humidified atmosphere containing 5% carbon dioxide (CO2) at 37° C. Thereafter, osteosarcoma cells were seeded onto the MC3T3 osteoblast cultured PCT at a concentration of 1×10⁵ cells and cultured under the same conditions as mentioned earlier. Subsequently, the PCT co-cultured with osteoblasts and osteosarcoma cells was transferred into a column for a dynamic culture system. Once connected to the dynamic culture system, the culture medium was allowed to flow into the column, where the co-cultured PCT was placed, at a flow rate of 5 ml/min. This dynamic culture setup was maintained for three days under a humidified atmosphere containing 5% carbon dioxide (CO2) at 37° C. The red circle in FIG. 34 denotes a cancer spheroid (osteosarcoma) that are imbedded inside of a normal cell (osteoblast) matrix mimicking an in vivo cancer microenvironment.

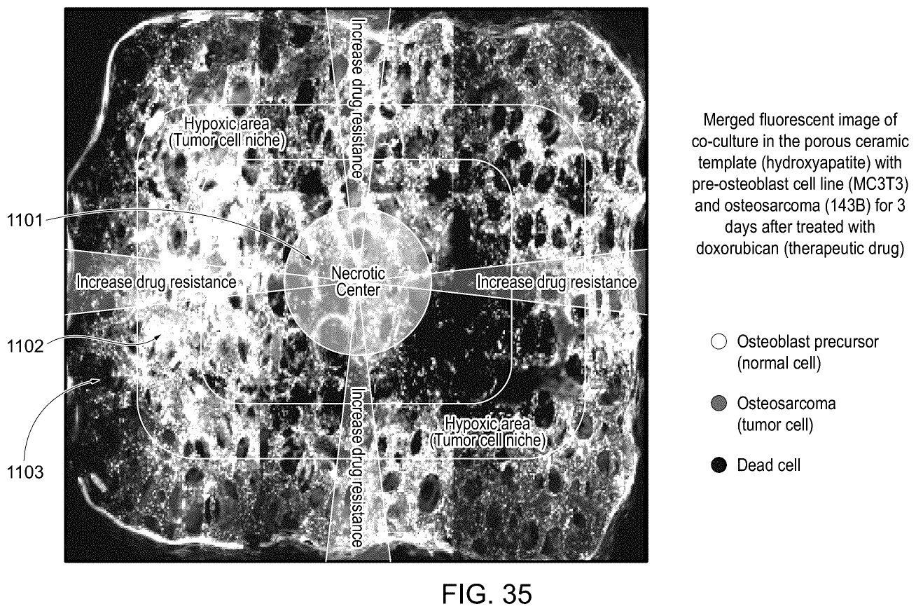

In an exemplary embodiment, FIG. 35 depicts a microenvironment of the 3D cancer model in the PCT illustrated in FIG. 34. Hypoxia is a crucial barrier to the delivery of chemotherapeutic agents. According to embodiments of the present disclosure, tumor hypoxia is induced by the characteristics of the template, resulting in a necrotic region 1101 near the center of the template. Within the necrotic center region 1101, most, if not all, of the cells, including cancer cells, are dead due to a lack of oxygen. The multi-leveled organization of the 3D porous ceramic template within a perfusion bioreactor system demonstrates deteriorated microenvironments. The reduction of drug concentration, nutrients, and oxygen creates a hypoxic area or environment (i.e., a tumor cell niche) in the middle region 1102, in which oxygen content is low, but good enough to keep cells alive. Low nutrition and acidosis incarnate in vivo tumor hypoxia. By virtue of this favorable microenvironment, the cancer cells are able to aggressively migrate and invade into the engineered bone-like porous ceramic template.

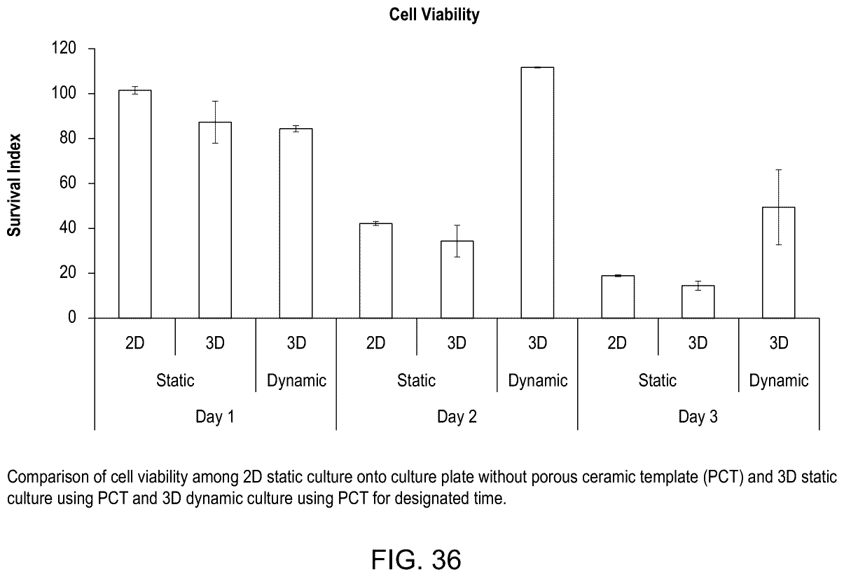

FIG. 36 depicts a comparison of cell viability among 2D static culture onto culture plate without a porous ceramic template (PCT), 3D static culture using a PCT, and 3D dynamic culture using a PCT for designated time. In this example, 2.5×10⁵ osteoblast precursor MC3T3 cells are deployed onto a 12 well culture plate for 2D static culture. 2.5×10⁵ MC3T3 cells are also seeded onto a PCT that is placed in a 12 well culture plate for 3D static culture. For the 3D dynamic culture, 2.5×10⁵ MC3T3 cells are seeded onto a PCT and held for one day under static culture condition then relocated into a culture column and subjected to dynamic culture using a peristaltic perfusion pump. In three days, cell viability is significantly reduced in both the 2D and 3D static culture conditions. Even though cell viability is significantly reduced under the 3D dynamic culture in comparison between day 1 and day 3, in comparing cell viability between static culture and dynamic culture, dynamic culture is significantly higher than that of static culture. Accordingly, the 3D dynamic culture system is more effective in recapitulating in vivo cellular microenvironment.

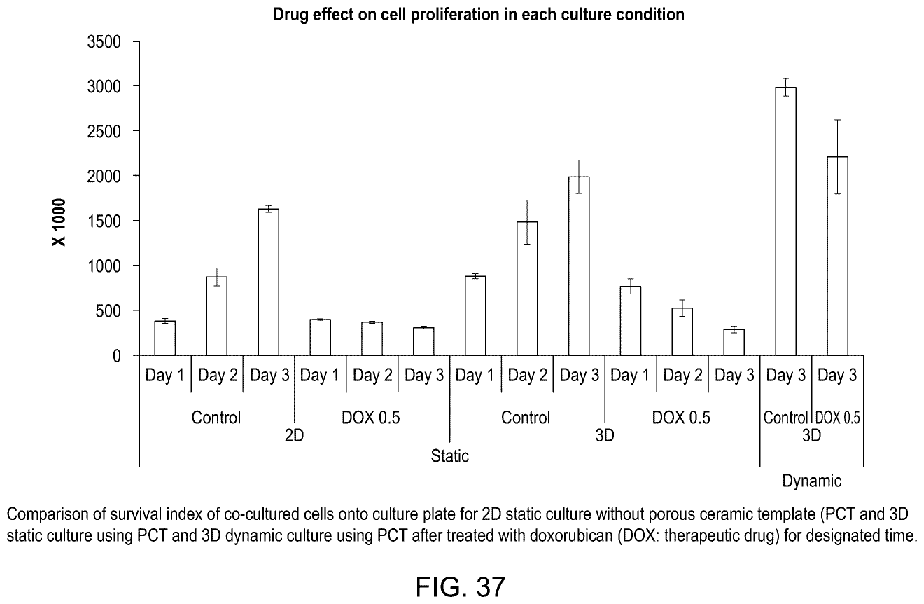

FIG. 37 depicts comparison of survival index of co-cultured cells onto a culture plate for 2D static culture without a porous ceramic template (PCT), 3D static culture using a PCT, and 3D dynamic culture using a PCT after treated with doxorubicin (DOX: therapeutic drug) for designated time. In this example, osteoblast precursor MC3T3 with 2.5×10⁵ cell population and osteosarcoma with 1×10⁵ cell population are deployed onto a 12 well culture plate for the 2D static culture. 2.5×10⁵ MC3T3 cell and 1×10⁵ osteosarcoma are also seeded onto a PCT that is placed in a 12 well culture plate for the 3D static culture. For the 3D dynamic culture, 2.5×10⁵ MC3T3 cell and 1×10⁵ osteosarcoma were seeded onto a PCT and held for two days under static culture condition, then relocated into a culture column and subjected to dynamic culture with doxorubicin (therapeutic agent) using a peristaltic perfusion pump. The 2D and 3D static culture conditions demonstrated the same trend of significant reduction of survival index associated with a 0.5 mg/ml concentration of doxorubicin treatment compared to control. This indicates that the 3D cell mass is not enough to drug resistance even in a 3D PCT model. On the other hand, the dynamic culture condition, a reduction of survival index with a 0.5 mg/ml concentration of doxorubicin, a 5 ml/min flow rate for five minutes per day treatment for three days was not significant compared to control. In comparison of cell survival index between 3D static culture and 3D dynamic culture, the 3D dynamic culture is significantly higher than the 3D static culture. In this regard, the 3D dynamic culture system is considered an effective alternative method of mimicking in vivo cancer model for performing a therapeutic agent test and/or screening.

In exemplary embodiments, the 3D cancel model of the present disclosure can be seeded with, or otherwise used in connection with, a patient's own tumor and/or normal cells, which can be cultured ex vivo. In this manner, the 3D cancer model of the present disclosure can be adapted for a specific patient for investigating a personalized cancel treatment agent or screening.

It will be understood that the embodiments described herein are merely exemplary and that a person skilled in the art may make many variations and modifications without departing from the spirit and scope of the invention. All such variations and modifications are intended to be included within the scope of the invention.

What is claimed is:

1. A tissue engineered three-dimensional model, comprising:
a three-dimensional porous ceramic template including a plurality of primary macro-pores; and
cells cultured in the three-dimensional porous ceramic template, wherein at least some of the cells form one or more three-dimensional cellular matrices in the primary macro-pores.

2. The three-dimensional model of claim 1, wherein the three-dimensional porous ceramic template includes micro-channels.

3. The three-dimensional model of claim 2, wherein the three-dimensional porous ceramic template includes a plurality of struts defining the primary macro-pores; wherein the micro-channels are formed in the struts and interconnected with each other for providing fluid flow therethrough; and wherein at least some of the normal cells are located in at least some of the micro-channels.

4. The three-dimensional model of claim 3, wherein the three-dimensional porous ceramic template includes sub-micro holes in surfaces thereof for providing additional locations for the cells to anchor.

5. The three-dimensional model of claim 1, wherein the cells include normal cells and tumor cells co-cultured in the three-dimensional porous ceramic template; wherein the normal cells form the three-dimensional cellular matrices in the primary macro-pores; and wherein the tumor cells are incapsulated in the three-dimensional cellular matrices.

6. The three-dimensional model of claim 5, wherein the three-dimensional porous ceramic template has a necrotic center region, hypoxic middle region, and normoxia outer region.

7. The three-dimensional model of claim 5, wherein the tissue engineered three-dimensional model exhibits expression of angiogenesis and vasculogenic mimicry features favoring tumor adaptation.

8. A process for preparing a tissue engineered three-dimensional model, comprising the steps of:
preparing a three-dimensional porous ceramic template including a plurality of primary macro-pores; and
culturing cells in the three-dimensional porous ceramic template, wherein at least some of the cells form one or more three-dimensional cellular matrices in the primary macro-pores.

9. The process of claim 8, wherein the three-dimensional porous ceramic template includes micro-channels.

10. The process of claim 9, wherein the three-dimensional porous ceramic includes a plurality of struts defining the primary macro-pores; wherein the micro-channels are formed in the struts and interconnected with one another for providing fluid flow therethrough; and wherein at least some of the normal cells are located in at least some of the micro-channels.

11. The process of claim 10, wherein the three-dimensional porous ceramic template includes sub-micro holes in surfaces thereof for providing additional locations for the cells to anchor.

12. The process of claim 8, wherein the culturing step includes the step of co-culturing normal cells and tumor cells in the three-dimensional porous ceramic template; wherein the normal cells form the three-dimensional cellular matrices in the primary macro-pores; and wherein the tumor cells are incapsulated in the cellular matrices.

13. The process of claim 12, wherein the three-dimensional porous ceramic template has a necrotic center region, hypoxic middle region, and normoxia outer region.

14. The process of claim 12, wherein the tissue engineered three-dimensional model exhibits expression of angiogenesis and vasculogenic mimicry features favoring tumor adaptation.

15. The process of claim 12, further comprising the step of testing a therapeutic agent for its effect on the normal cells and/or the tumor cells.

16. The process of claim 15, wherein the testing step includes the steps of applying the therapeutic agent to the tissue engineered three-dimensional model.

17. The process of claim 16, wherein the applying step includes the step of placing the tissue engineered three-dimensional model in a bioreactor system connected to a medium vessel, the medium vessel including a medium in which the therapeutic agent is dissolved.

18. An apparatus, comprising:
a tissue engineered three-dimensional model, including:
a three-dimensional porous ceramic template including a plurality of primary macro-pores; and
cells cultured in the three-dimensional porous ceramic template, wherein at least some of the cells form one or more three-dimensional cellular matrices in the primary macro-pores; and
a bioreactor system including:
a culture column configured to receive the three-dimensional porous ceramic template;
a culture medium vessel operably connected to the culture column;
a peristaltic perfusion pump operably connected to the culture column and to the culture medium vessel, wherein the peristaltic perfusion pump is configured to apply a dynamic culture system within the culture column.

19. The apparatus of claim 18, wherein the culture medium vessel is filled with a medium in which a therapeutic agent is dissolved.

20. The apparatus of claim 19, wherein the therapeutic agent is selected from a group consisting of Doxorubicin, Cisplatin, Methotrexate, Ifosfamide, Actinomycin D, Bleomycin, Vincristine, Cyclophosphamide, Paclitaxel, Carboplatin, any kind of therapeutic agent developed or under development for cancer/tumor treatment, and a combination of two or more of the foregoing agents.
